(19) United States
(12) Patent Application Publication
(10) Pub. No.: US 2024/0325451 A1
(43) Pub. Date: Oct. 3, 2024

(54) METHODS OF INDUCING ORGAN TRANSPLANTATION IMMUNE TOLERANCE AND ASSOCIATED COMPOSITIONS AND METHODS

(71) Applicant: CITY OF HOPE, Duarte, CA (US)

(72) Inventor: Defu ZENG, Duarte, CA (US)

(73) Assignee: CITY OF HOPE, Duarte, CA (US)

(21) Appl. No.: 18/622,589

(22) Filed: Mar. 29, 2024

Related U.S. Application Data
(60) Provisional application No. 63/493,408, filed on Mar. 31, 2023.

(51) Int. Cl. A61K 35/28; A61K 31/675; A61K 31/7056; A61K 35/12; A61K 35/26; A61K 39/395; A61L 27/36; A61L 27/54; A61P 37/06

(52) U.S. Cl. CPC A61K 35/28; A61K 31/675; A61K 31/7056; A61K 35/26; A61K 39/39541; A61L 27/3604; A61L 27/54; A61P 37/06; A61K 2035/122; A61K 2035/124

(57) ABSTRACT

Described herein are methods and transplant compositions for promoting or inducing organ transplant tolerance and/or immune tolerance by conditioning a recipient with radiation-free, low-doses of cyclophosphamide (CY), pentostatin (PT), and anti-thymocyte globulin (ATG) prior to transplantation of PD-L1+ donor-derived CD4+ T-depleted bone marrow cells. In certain embodiments, the methods may also include transplanting an organ, such as a solid organ, into the recipient. The transplant compositions may comprise a therapeutically effective amount of PD-L1+ donor-derived CD4+ T-depleted bone marrow cells and a donor organ.

### CROSS-REFERENCE TO RELATED APPLICATIONS

This application claims the benefit of U.S. Provisional Patent Application No. 63/493,408 filed Mar. 31, 2023. The contents this provisional application are incorporated herein by reference in their entirety.

### BACKGROUND

A radiation-free regimen for induction of full major histocompatibility complex (MHC)-mismatched mixed chimerism (MC) in autoimmune mice, which consists of non-myeloablative conditioning with low-dose cyclophosphamide (CY), pentostatin (PT), and anti-thymocyte globulin (ATG) as well as infusion of donor CD4⁺ T-depleted hematopoietic grafts has been developed and is referred to as COH-MC-17. While a phase I trial with sickle cell subjects (NCT03249831) using the COH-MC-17 regimen in the absence of organ transplantation is ongoing, stable full HLA-mismatched MC and organ transplantation has not been achieved in humans.

Tissue PD-L1 interaction with PD-1 on activated T cells play important roles in down-regulating autoimmunity, tumor immunity, graft versus host disease (GVHD), and organ transplant rejection. Whether PD-L1 expressed by donor-type hematopoietic cells and solid organs is involved with induction of MC and MC-mediated organ transplant immune tolerance has not been evaluated.

Induction of MC via allogeneic hematopoietic cell transplantation (HCT) has been proposed for organ transplantation immune tolerance, however, since clinical organ transplantation often uses organs from deceased donors that are fully HLA-mismatched, stable full HLA-mismatched MC and organ transplantation remains a barrier to clinical application. Accordingly, there remains a need to develop effective methods to induce organ transplantation immune tolerance to induce stable full HLA-mismatched MC and organ transplantation in humans.

### SUMMARY

In some embodiments, the present technology relates to a methods and compositions for promoting or inducing organ transplant tolerance in a recipient, including but not limited to, immune tolerance, central immune tolerance, or peripheral immune tolerance. In some embodiments, the methods comprise a solid organ, including but not limited to heart, lung, liver, kidney, intestine, pancreas, eye, or skin. In some embodiments, the method comprises (a) administering a conditioning regimen comprising low-doses of cyclophosphamide (CY), pentostatin (PT), and anti-thymocyte globulin (ATG) to the recipient; (b) transplanting a therapeutically effective amount of PD-L1⁺ donor-derived CD4⁺ T-depleted bone marrow cells into the recipient; and (c) transplanting an organ into the recipient. In some embodiments, the method comprises transplanting a therapeutically effective amount of PD-L1⁺ CD4⁺ T-depleted donor bone marrow cells into the recipient conditioned with a regimen comprising low-doses of CY, PT, and ATG to the recipient. In some aspects, an organ is transplanted into the recipient. In some aspects, the PD-L1⁺ donor-derived CD4⁺ T-depleted bone marrow cells include donor-derived CD4⁺ T-depleted spleen cells, and donor-derived CD4⁺ T-depleted bone marrow cells. In some aspects, expression of PD-L1 on the PD-L1⁺ donor-derived CD4⁺ T-depleted bone marrow cells is determined prior to the transplanting of (b). In some aspects, the PD-L1⁺ donor-derived CD4⁺ T-depleted bone marrow cells are selected based on PD-L1⁺ expression prior to the transplanting of (b). In some aspects, the PD-L1⁺ donor-derived CD4⁺ T-depleted bone marrow cells are enriched for PD-L1⁺ expression prior to the transplanting of (b). In some aspects, (c) occurs before, during, or after (a) and (b). In some aspects, the conditioning regimen of is administered to the recipient before transplantation of the population of PD-L1⁺ donor-derived bone marrow cells in (b). In some aspects, CY, PT, and ATG are administered simultaneously. In some aspects, a population of PD-1⁺ T cells is present in the recipient before, during, or after any of (a), (b), or (c) are performed. In some aspects,

In some embodiments, the present technology relates to a method of promoting or inducing immune tolerance in an organ transplant recipient, the method comprising (a) administering a conditioning regimen comprising low-doses of CY, PT, and ATG to the recipient; (b) measuring PD-L1 expression on a population of donor-derived CD4⁺ T-depleted donor marrow cells; (c) selecting a population of PD-L1⁺ donor-derived CD4⁺ T-depleted bone marrow cells from the population in (b); and (d) transplanting a therapeutically effective amount of PD-L1⁺ donor-derived CD4⁺ T-depleted bone marrow cells selected in (c) into the recipient. In some aspects, an organ is transplanted into the recipient.

In some embodiments, the methods in accordance with the present technology provide for population of donor-derived PD-L1⁺ CD8⁺ dendritic cells present in the recipient after organ transplant tolerance has been established. In some aspects, the population of donor-derived PD-L1⁺ CD8⁺ dendritic cells is derived from the transplanted bone marrow cells. In some aspects, a population of recipient peripheral T regulatory cells is present in the recipient after engraftment of the transplanted bone marrow cells. In some aspects, the population of recipient peripheral T regulatory cells expands in the recipient after organ transplant tolerance has been established. In some aspects, the administration of the conditioning regimen and transplantation of the PD-L1⁺ donor-derived CD4⁺ T-depleted bone marrow cells induces stable mixed chimerism in the recipient. In some aspects, the stable mixed chimerism is haploidentical stable mixed chimerism. In some aspects, the donor is haploidentical to the recipient. In some aspects, the donor is haplo-mismatched to the recipient. In some aspects, the donor is not full-HLA- or MHC-matched to the recipient. In some aspects, the donor is living or deceased. In some aspects, the conditioning regimen is radiation free. In some aspects, the conditioning regimen is non-myeloablative.

In some aspects, the recipient is a human and the daily dose for CY is from about 25 to about 750 mg/kg/day, the daily dose for PT is from about 2 mg/m²/dose to about 8 mg/m²/dose, and the dose for ATG is from 1.0 mg/kg to about 8.0 mg/kg. In some aspects, the daily dose for CY is from about 25 mg to about 750 mg, the dose for PT is from about 2 mg/m²/dose to about 8 mg/m²/dose, and the dose for ATG is from 1.0 mg/kg to about 8.0 mg/kg.

In some embodiments, the methods of the present technology comprise administration of a population of conditioning cells that facilitate engraftment during hematopoietic cell transplantation (HCT). In some aspects, the population of conditioning cells that facilitate engraftment during HCT is selected from one or more populations of conditioning donor cells selected from donor CD4⁺ T-depleted spleen cells, donor CD8⁺ T cells, and donor Granulocyte colony-stimulating factor (G-CSF)-mobilized peripheral blood mononuclear cells. In some aspects, the transplantation of the population of donor bone marrow cells occurs on the same day as or after the administration of the population of conditioning cells that facilitate engraftment during HCT. In some aspects, the population of conditioning donor cells, the population of donor bone marrow cells, or both are MHC- or HLA-mismatched to the recipient. In some embodiments, selecting a population of PD-L1⁺ donor-derived CD4⁺ T-depleted bone marrow cells comprises enriching for the population of PD-L1⁺ cells from the CD4⁺ T-depleted bone marrow cells, and optionally isolating the population of PD-L1⁺ cells from the CD4⁺ T-depleted bone marrow cells.

In some embodiments, the present technology relates to compositions for promoting or inducing organ transplant tolerance or immune tolerance in a recipient. In some embodiments, the composition is a transplant composition. In some embodiments, the organ is a solid organ including, but not limited to, heart, lung, liver, kidney, intestine, pancreas, eye, or skin. In some embodiments, the transplant composition comprises a first composition and a second composition, the first composition comprising or consisting of therapeutically effective amount of PD-L1⁺ donor-derived CD4⁺ T-depleted bone marrow cells and the second composition comprising or consisting of the donor organ. In some aspects, the expression of PD-L1 on the PD-L1⁺ donor-derived CD4⁺ T-depleted donor bone marrow cells is determined prior to the transplanting of the transplant composition into the recipient. In some aspects, the PD-L1⁺ donor-derived CD4⁺ T-depleted donor bone marrow cells include donor-derived CD4⁺ T-depleted spleen cells, and donor-derived CD4⁺ T-depleted bone marrow cells. In some aspects, the PD-L1⁺ donor-derived CD4⁺ T-depleted bone marrow cells are selected based on PD-L1⁺ expression prior to the transplanting of the transplant composition into the recipient. In some aspects, the PD-L1⁺ donor-derived CD4⁺ T-depleted bone marrow cells are enriched for PD-L1⁺ expression prior to the transplanting of the transplant composition into the recipient.

In some embodiments, the transplant composition comprises a conditioning regimen comprising low-doses of CY, PT, and ATG administered to the recipient. In some aspects, the conditioning regimen is administered to the recipient before the therapeutically effective amount of PD-L1⁺ donor-derived CD4⁺ T-depleted bone marrow cells and before the donor organ. In some aspects, the conditioning regimen is administered to the recipient after the therapeutically effective amount of PD-L1⁺ donor-derived CD4⁺ T-depleted bone marrow cells and before the donor organ. In some aspects, the conditioning regimen is administered to the recipient after the therapeutically effective amount of PD-L1⁺ donor-derived CD4⁺ T-depleted bone marrow cells and after the donor organ. In some aspects, a population of recipient peripheral T regulatory cells is present in the recipient after engraftment of the transplanted bone marrow cells. In some aspects, the population of recipient peripheral T regulatory cells expands in the recipient after organ transplant tolerance has been established. In some aspects, the donor is haploidentical to the recipient, haplo-mismatched to the recipient, or is not full-HLA- or MHC-matched to the recipient.

In some embodiments, the transplant composition is used to promote or induce immune tolerance in a recipient. In some embodiments, the transplant composition is used to promote or induce organ transplant tolerance in a recipient. In some aspects, a population of donor-derived PD-L1⁺ CD8⁺ dendritic cells is present in the recipient after organ transplant tolerance has been established. In some embodiments, the population of donor-derived PD-L1⁺ CD8⁺ dendritic cells is derived from the transplanted bone marrow cells.

### BRIEF DESCRIPTION OF THE DRAWINGS

FIGS. 1A-11 illustrate that induction of MHC (human leukocyte antigen (HLA))-mismatched mixed chimerism (MC) using a conditioning regimen (“conditioning-induced MCs”) in accordance with the present technology establishes immune tolerance for donor organs transplanted before and after MC induction. FIGS. 1A and 1B illustrate the experimental scheme for heart transplantation (HTX) or skin transplantation (STX) from BALB/c donors performed before conditioning on day 12 (FIG. 1A) or after HCT on day 30 (FIG. 1B). FIG. 1C shows survival in HTX mice before conditioning (HTX Before Cond), after HCT (HTX after HCT), or conditioning alone (Cond Alone). n=4-6, *** P<0.001, using log-rank test.

FIG. 1D provides representative histopathology micrographs (100× magnification) of the mouse heart grafts HTX Before Cond, HTX After HCT, or Cond Alone, n=4-6 samples/group. FIG. 1E illustrates pathology scores (mean±SEM) for Cond Alone, HTX before Cond, and HTX After HCT. n=4-6 samples/group. FIG. 1F depicts survival in STX mice STX before conditioning (STX Before Cond), after HCT (STX after HCT), or Cond Alone. n=6, **** P<0.0001, using log-rank test. FIG. 1G. provides representative photos and histopathology micrographs (100× magnification) of the mouse skin grafts STX Before Cond or STX After HTC compared to those receiving Cond Alone. n=6 samples/group. FIG. 1H illustrates pathology scores (mean±SEM) for Cond Alone, STX before Cond, and STX After HCT. n=4-6 samples/group FIG. 1I depicts CD8⁺ and CD4⁺ T cell counts in STX mice (MCs and control) on day 30 or day ≥60. A representative histogram and mean±SEM are shown for 4 replicates. *** P<0.001, using one-way ANOVA.

FIGS. 2A-2D illustrate that MCs induced with a conditioning regimen in accordance with the present technology provide immune tolerance for skin graft transplanted during induction of MC but rejects third-party skin graft. FIG. 2A provides representative flow cytometry patterns and mean±SEM percentage of donor-type and host-type TCR-β⁺ T cells, B220⁺ B cells, and Mac1⁺/Gr1⁺ myeloid cells in peripheral blood (PB), spleen (SPL), and bone marrow (BM) samples on day 100 after HCT. n=4. FIG. 2B exhibits survival Cond Alone with STX conditioning without HCT, and MC mice with STX after conditioning during HCT. n=6. *** P<0.001, using log-rank test. FIG. 2C illustrates representative skin graft photographs and H&E staining histopathology micrographs of mice receiving either Cond Alone or STX during HCT. (100× magnification), n=6. FIG. 2D depicts MC survival receiving donor STX or third party STX on day 60 after HCT. n=6. *** P<0.001, using log-rank test. Each result is combined from 3-4 independent experiments. Comparison of survival curves is using log-rank test. *** P<0.001.

FIGS. 3A-3G illustrate that MCs with MHC knockout (MHCII^{-/-}) donor hematopoietic cells induced with a conditioning regimen in accordance with the present technology may provide tolerance to donor type MHCII^{-/-} but not MHC WT (MHCII^{+/+}) heart graft or skin graft. FIG. 3A illustrates the experimental scheme, where either CD45.1 B6 or BALB/c were conditioned and given HCT from MHCII^{-/-} BALB/c or B6 donors respectively and transplanted with either MHCII^{-/-} or WT donor heart or skin graft on day 30 after HCT.

FIG. 3B provides representative flow cytometry patterns of donor-type and host-type TCR-β⁺ T cells, B220⁺ B cells, and Mac1⁺/Gr1⁺ myeloid cells in PB, SPL, and BM samples on day 100 after HCT for MHCII^{-/-} MC under the experimental scheme illustrated in FIG. 3A. n=5. FIG. 3C illustrates survival after HTX from MHCII^{-/-} or WT donor BALB/c hearts. n=4-5. P<0.01, using log-rank test. FIG. 3D depicts representative H&E staining histopathology micrographs of HTX from MHCII^{-/-} or WT donor BALB/c hearts (100× magnification), n=4-5. FIG. 3E illustrates survival following STX from MHCII^{-/-} or WT donor BALB/c skin. n=5. P<0.01, using log-rank test. FIG. 3F represents skin graft photographs and H&E staining histopathology microphotographs following STX from MHCII^{-/-} or WT donor BALB/c skin. (100× magnification), n=5. FIG. 3G illustrates survival for B6 mice given conditioning alone (no HCT) or MCs given donor WT BALB/c hematopoietic cells after conditioning, and then subjected to MHCII^{-/-} BALB/c STX 30 days after conditioning. n=5. Each result is combined from 3 independent experiments. ** P<0.01, using log-rank test.

FIGS. 4A-4H illustrate the conditioning-induced MCs of the present technology and tolerizing donor type MHCII^{-/-} organs may not require donor hematopoietic cell MHCII. FIG. 4A illustrates the experimental scheme: CD45.1 B6 recipients were conditioned and followed by HCT from MHCII^{-/-} or WT BALB/c donors on day 0 to induce MHCII^{-/-} or WT MC and then transplanted with either MHCII^{-/-} or WT donor heart or skin graft on day 30 after HCT. FIG. 4B provides representative flow cytometry patterns and mean±SEM of percentage of donor-type and host-type TCR-β+ T, B220+ B, and Mac1+/Gr1+ myeloid cells in PB, SPL, and BM on day 100 after HCT, n=5. FIG. 4C demonstrates survival after HTX following transplantation with tissues provided in FIG. 4A. n=5, P<0.01, using log-rank test. FIG. 4D provides representative histopathology micrographs of heart grafts following transplantation derived from mice shown in FIG. 4A. (100× magnification), n=5. FIG. 4E depicts pathology scores (mean±SEM) of HTX among experimental groups, n=5. FIG. 4F illustrates survival following STX derived from mice shown in FIG. 4A. n=6. P<0.0001, using log-rank test. FIG. 4G illustrates representative skin grafts and histopathology micrographs following transplantation derived from mice shown in FIG. 4A. (100× magnification), n=6. FIG. 4H illustrates pathology scores (mean±SEM) of STX among experimental groups, n=6. Each result is combined from 3-4 independent experiments. Survival curves compared using log-rank test, means compared using unpaired two-tailed Student's t test. *, **, *** P<0.05, 0.01, 0.001.

FIGS. 5A-5D illustrate that residual donor MHC-reactive CD4⁺ and CD8⁺ T cells may become anergic or exhausted in WT conditioning-induced MCs of the present technology. FIGS. 5A and 5B provide representative flow cytometry patterns and mean±SEM percentage of CD62L⁻CD44⁺CD4⁺ Tem cells among host-type CD4⁺ T cells and CD73^{hi}FR4^{hi} anergic CD4⁺ T cell subset among host-type Foxp3-CD4⁺ Tem cells (FIG. 5A), and CD62L⁻CD44⁺CD4⁺ Tem cells among host type CD8⁺ T cells and Ly108-CD39⁺ exhausted CD8⁺ T cell subset among KLRG1·PD-1⁺ CD8⁺ Tem cells (FIG. 5B) without STX. n=4-14, using one-way ANOVA. FIGS. 5C and 5D provide representative flow cytometry patterns and mean±SEM of percentage of subsets shown in recipients of FIGS. 5A and 5B with STX. FIG. 5C illustrates mean±SEM percentage of CD62L⁻CD44⁺CD4⁺ Tem cells among host-type CD4⁺ T cells and CD73^{hi}FR4^{hi} anergic CD4⁺ T subset among host-type Foxp3⁻CD4⁺ Tem cells, and FIG. 5D depicts the same measurement in CD62L⁻CD44⁺CD4⁺ Tem cells among host-type CD8⁺ T cells and Ly108⁻CD39⁺ exhausted CD8⁺ T subset among KLRG1⁻PD-1⁺ CD8⁺ Tem cells. n=4-5. Each result is combined from 3-4 independent experiments. Means compared using one-way ANOVA. *, **, ***, P<0.05, 0.01, 0.001.

FIGS. 6A-6C illustrate that residual donor MHCII-reactive CD4⁺ T cells in WT MCs induced with a conditioning regimen in accordance with the present technology differentiate into Helios⁻NRP1⁺pTreg cells. FIG. 6A provides representative flow cytometry patterns and mean±SEM of percentage of NRP1⁺ precursors of Treg cells among anergic Foxp3⁻CD4⁺ Tcon cells in the absence or presence of STX. N=5-14. FIGS. 6B and 6C provide representative of flow cytometry patterns and mean±SEM of percentage of Foxp3⁺CD4⁺ Treg cells among host-type CD62L⁻CD44⁺CD4⁺ Tem cells, Helios+ tTreg cells among total Treg cells, and NRP1⁺ pTreg cells among Helios⁻ Treg cells in the absence (FIG. 6B) or presence (FIG. 6C) of skin graft, n=4-14. Each result is combined from 3-4 independent experiments. Means compared using unpaired two-tailed Student's t test. *, * P<0.05, 0.01

FIGS. 7A-7D illustrate that CD62L⁻CD44⁺ Tem and anergic/exhausted of CD4⁺ & CD8⁺ T cells compared between absence and presence of STX. FIGS. 7A and 7B illustrate mean±SEM of percentage of CD62L⁻CD44⁺CD4⁺ Tem cells among host-type CD4⁺ T cells and anergic CD73^{hi}FR4^{hi}CD4⁺ T subset among host-type Foxp3⁻CD4⁺ Tem cells in the absence or presence of skin graft (W/O Skin or W/T Skin), n=4-14. FIGS. 7C and D depict mean±SEM of percentage of CD62L⁻CD44⁺CD8⁺ Tem cells among host type CD8⁺ T cells and exhausted Ly108⁻CD39⁺CD8⁺ T subset among KLRG1⁻PD-1⁺ CD8⁺ Tem cells in the absence or presence of donor-type skin graft (W/O Skin and W/T Skin), n=4-14. Each result is combined from 3-4 independent experiments. Multiple means compared using one-way ANOVA. **, *** P<0.01, 0.001.

FIGS. 8A-8D illustrate the yield comparison of pTreg precursor, total Treg, tTreg, and pTreg cells in WT MC and MHCII^{-/-}MC with or without donor-type STX. FIG. 8A shows yield of NRP1+ pTreg precursors in the absence or presence of STX, n=5-14. FIGS. 8B and C illustrate yield of Foxp3⁺CD4⁺ total Treg cells, Helios+ tTreg cells, and Helios⁻NRP1⁺ pTreg cells in the absence (FIG. 8B) or presence (FIG. 8C) of STX, n=4-14. FIG. 8D shows yield of total Treg, tTreg, and pTreg in the WT MC with or without donor-type STX (W/O Skin or W/T Skin), n=5-7. Each result is combined from 3-4 independent experiments. Means compared using unpaired two-tailed Student's t test. *, **, *** P<0.05, 0.01, 0.001.

FIGS. 9A and 9B illustrate donor MHCII augments thymic generation of host-type tTreg cells in MCs induced with a conditioning regimen of the present technology. CD4⁺CD8 thymocytes from WT and MHCII^{-/-} MCs were analyzed with flow cytometry for percentage of tTreg cells. FIGS. 9A and 9B provide representative flow cytometry patterns (FIG. 9A) and mean±SEM percentage (FIG. 9B) of Foxp3⁺ Treg cells among host-type and donor-type CD4⁺CD8 thymocytes in the absence of STX on day ≥60 after HCT for different groups. Result is combined from 3 independent experiments. Means compared using unpaired two-tailed Student's t test. ** P<0.01.

FIGS. 10A-10E demonstrate depletion of host-type Treg cells may result in reduction of donor-type CD8⁺ DCs and their PD-L1 expression levels and rejection of donor-type STX. FIG. 10A illustrates the experimental schematic for mice which express the diphtheria toxin receptor (DTR) gene from the Foxp3 locus (Foxp3^{DTR}) or WT B6 mice with conditioning-induced MCs. FIG. 10B illustrates survival following STX in mice as shown in FIG. 10A. n=5-8, P<0.0001, using log-rank test. FIG. 10C shows representative flow cytometry patterns and mean±SEM percentage of donor-type TCR-β⁺ T cells, B220⁺ B cells, and Mac1⁺/Gr1⁺ myeloid cells in the PB before and after DT treatment, n=4. FIG. 10D provides representative flow cytometry patterns and mean±SEM percentage of donor-type CD8⁺CD11b⁻CD11c⁺ cells (CD8⁺ DCs) and CD8⁺CD11b⁻CD11c⁺ cells (CD11b⁺ DCs) in SPL of DTR MC+PBS mice and DTR MC+DT mice. n=4, using unpaired two-tailed Student's t test. FIG. 10E shows representative histogram patterns and mean±SEM for mean fluorescence intensity (MFI) of PD-L1 and MHCII I-A/I-E expressions among donor-type CD8⁺ DCs and CD11b⁺ DCs in SPL of DTR MC+PBS and DTR MC+DT mice at 30 days after treatment. n=4, Each result is combined from 3 independent experiments. Survival curves compared using log-rank test. Means compared using unpaired two-tailed Student's t test. *, **, *** P<0.05, 0.01, 0.001.

FIGS. 11A-11C illustrate that depletion of host-type Foxp3⁺ Treg cells may not interfere with MC status in conditioning-induced MCs. FIG. 11A provides representative flow cytometry patterns and mean±SEM percentage of Foxp3⁺ Treg cells among host-type CD62L⁻CD44⁺CD4⁺ T cells in the SPL, draining LN, skin, and PB of DTR MC+PBS and DTR MC+DT mice. n=4. FIG. 11B illustrates representative flow cytometry patterns and mean±SEM percentage of Foxp3⁺ Treg cells among donor-type CD62L⁻CD44⁺CD4⁺ T cells in the SPL, LN, skin, and PB from Foxp3^{DTR-KI} MCs treated with DT or PBS, n=4. FIG. 11C depicts representative flow cytometry patterns and mean±SEM of percentage of donor TCR-β⁺ T cells, B220⁺ B cells, and Mac1⁺/Gr1⁺ myeloid cells in SPL before and after DT treatment. n=4. Each result is combined from 3 independent experiments. Means compared using unpaired two-tailed Student's t test or one-way ANOVA. *, **, *** P<0.05, 0.01, 0.001.

FIGS. 12A-12F illustrate that PD-L1 expressed by donor hematopoietic cells and PD-1 expressed by host cells may be required for the expansion of host-type Helios⁻NRP1⁺ pTreg cells and skin graft tolerance in conditioning-induced MCs. FIG. 12A illustrates survival following STX for WT MC, PD-L1^{-/-}Donor MC, and PD-1^{-/-}Rec MC mice. n=5-7. P<0.01. FIGS. 12B and 12C provide mean±SEM percentage of TCR-β⁺ T cells, B220⁺ B cells, and Mac1⁺/Gr1⁺ myeloid cells in PB and SPL. n=4-5. FIG. 12D illustrates mean±SEM yield of mononuclear cells (MNC) in the skin for WT MC, PD-L1^{-/-} Donor MC, and PD-1^{-/-}Rec MC mice. n=3-5, using one-way ANOVA. FIG. 12E illustrates representative flow cytometry patterns and mean±SEM percentage of host-type CD62L⁻CD44⁺CD4⁺ and CD62L⁻CD44⁺ CD8⁺ Tem cells in the skin for WT MC, PD-L1^{-/-}Donor MC, and PD-1^{-/-}Rec MC mice, n=3-5, using one-way ANOVA. FIG. 12F illustrates representative flow cytometry patterns and mean±SEM percentage of Foxp3⁺CD4⁺ total Treg cells among CD62L⁻CD44⁺CD4⁺ Tem cells, Helios⁺ tTreg cells among total Treg cells, and NRP1⁺ pTreg cells among Helios⁻ Treg cells in the SPL for WT MC, PD-L1^{-/-}Donor MC, and PD-1^{-/-}Rec MC mice. n=3-5. Results are 3 combined independent experiments. Means compared using unpaired two-tailed Student's t test or one-way ANOVA. *, **, *** P<0.05, 0.01, 0.001

FIGS. 13A-13C demonstrate that lack of donor hematopoietic cells PD-L1 interaction with PD-1 on host T cells may permit conditioning-induced MCs. FIGS. 13A-13C show representative flow cytometry patterns of donor-type and host-type T cells TCR-β⁺ T cells, B220⁺ B cells, and Mac1⁺/Gr1⁺ myeloid cells in PB and SPL of WT MC (FIG. 13A), PD-L1^{-/-}Donor MC (FIG. 13B), and PD-1/Rec MC (FIG. 13C) mice. n=4-5.

FIG. 14 illustrates that PD-L1^{-/-}conditioning-induced MCs of the present technology transplanted with PD-L1^{-/-}donor hematopoietic cells reject PD-L1^{-/-}donor STX. Survival following STX is illustrated for both WT MC+WT skin and PD-L1^{-/-} MC+PD-L1^{-/-} skin mice (n=3-5). Result is 3 combined independent experiments. Survival curves combined using log-rank test. ** P<0.01.

FIGS. 15A-15G illustrate that PD-L1 expressed by donor-type graft may be required for the expansion of host-type NRP1⁺ pTreg cells and graft tolerance in MCs induced with a conditioning regimen in accordance with the present technology. FIGS. 15A and 15B provide survival following HTX (P<0.001, using log-rank test) (FIG. 15A) and representative histopathology micrographs of heart grafts in WT Heart and PD-L1^{-/-}Heart mice (100× magnification) and pathology scores (mean±SEM), n=4 (FIG. 15B). FIGS. 15C and 15D illustrate survival following STX (P<0.0001, using log-rank test) (FIG. 15C) and representative histopathology micrographs in WT Skin and PD-L1/Skin mice (100× magnification) and pathology scores (mean±SEM) (FIG. 15D) for the experimental groups, n=5. FIG. 15E provides representative flow cytometry patterns and mean±SEM percentage of donor-type TCR-β⁺ T cells, B220⁺ B cells, and Mac1⁺/Gr1⁺ myeloid cells in PB in WT MCs before and after PD-L1/STX when graft is being rejected around day 10. n=5. FIGS. 15F and 15G show mean±SEM percentage of Foxp3⁺CD4⁺ total Treg cells among CD62L CD44⁺CD4⁺ Tem cells, Helios+ tTreg cells among total Treg cells, and NRP1⁺ pTreg cells among Helios⁻ Treg cells in SPL (FIG. 15F) and draining LN (FIG. 15G) on day 10 after STX. n=5-8. P<0.05, P<0.01, P<0.001, using unpaired two-tailed Student's t test. Results are combined from 3 independent experiments. Survival curves compared using log-rank test; means compared using unpaired two-tailed Student's t test. *, **, *** P<0.05, 0.01, 0.001.

FIG. 16 illustrates a yield comparison of Treg and pTreg cells in the spleen of WT MC, PD-L1^{-/-}Donor MC or PD-1/Rec MC groups. n=3-5. Each result is combined from 3 independent experiments. Comparison of multiple means is using one-way ANOVA. *, ** P<0.05, 0.01.

FIGS. 17A-17C illustrate that PD-L1 expressed by donor-type graft may be required for the expansion of host-type NRP1⁺ pTreg cells in MCs induced with a conditioning regimen in accordance with the present technology. FIG. 17A depicts representative flow cytometry patterns and mean±SEM percentage of donor-type TCR-β⁺ T cells, B220⁺ B cells, and Mac1⁺/Gr1⁺ myeloid cells in SPL from PD-L1/Skin mice and WT Skin mice after HCT, n=5-8. FIGS. 17B and 17C depict representative flow cytometry patterns of Foxp3⁺CD4⁺ total Treg cells among host-type CD62L⁻CD44⁺CD4⁺ Tem cells, Helios⁺ tTreg cells among total Treg cells, and NRP1⁺ pTreg cells among Helios⁻ Treg cells in the SPL (FIG. 17B) and LN (FIG. 17C) for WT MC group subjected to either WT or PD-L1^{-/-} STX on day 60 after HCT, n=5-8.

FIGS. 18A-18D show a yield comparison of total CD4⁺ Tcon, total Treg, tTreg, and pTreg in MCs given WT and PD-L1/skin graft. FIGS. 18A and B display yield of Foxp3-CD4⁺ Tcon in SPL (FIG. 18A) and LN (FIG. 18B) on day 10 after STX, n=5-8. FIGS. 18C and 18D illustrate yield of Foxp3⁺CD4⁺ total Treg cells, Helios+ tTreg cells, and Helios-NRP1+ pTreg cells in SPL (FIG. 18C) and LN (FIG. 18D), n=5-8. Each result is combined from 3 independent experiments. Means compared using unpaired two-tailed Student's t test. * P<0.05.

FIGS. 19A-19D illustrate that PD-L1 expressed by donor-type graft may be required for the expansion of host-type pTreg cells and graft tolerance during induction of MCs induced with a conditioning regimen of the present technology. FIG. 19A provides survival after STX and representative H&E staining histopathology microphotographs of skin grafts in WT Skin and PD-L1^{-/-} Skin mice (100× magnification) for rejected PD-L1^{-/-} Skin mice and tolerant WT Skin mice. n=5-8. **** P<0.0001, using log-rank test. FIG. 19B illustrates representative flow cytometry patterns and mean±SEM percentage of donor-type TCR-β⁺ T cells, B220⁺ B cells, and Mac1⁺/Gr1⁺ myeloid cells in the SPL from PD-L1^{-/-}Skin and WT Skin mice. n=5-8. FIG. 19C illustrates mean±SEM percentage of CD62L⁻CD44⁺CD4⁺ and CD62L⁻CD44⁺CD8⁺ Tem cells among host-type CD4⁺ and CD8⁺ T cells in SPL and LN from WT MC group subjected to either WT Skin or PD-L1/Skin STX during HCT for different groups, n=5. FIG. 19D depicts mean±SEM percentage of Foxp3⁺CD4⁺ Treg cells among host-type CD62L⁻CD44⁺CD4⁺ Tem cells, Helios+ tTreg cells among total Treg cells, and NRP1⁺ pTreg cells among Helios⁻ Treg cells in the SPL and LN for WT Skin or PD-L1/Skin mice. n=5. * P<0.05, ** P<0.01, using unpaired two-tailed Student's t test. Each result is combined from 3 independent experiments.

### DETAILED DESCRIPTION

The following description of the present technology is merely intended to illustrate various embodiments of the present technology. As such, the specific modifications discussed are not to be construed as limitations on the scope of the present technology. It will be apparent to one skilled in the art that various equivalents, changes, and modifications may be made without departing from the scope of the present technology, and it is understood that such equivalent embodiments are to be included herein.

The present technology comprises various methods for promoting or inducing organ transplant tolerance which include establishing stable mixed chimerism without inducing GVHD. According to the embodiments of the present technology, the methods for promoting or inducing organ transplant tolerance in a recipient include administering a radiation-free non-myeloablative conditioning regimen comprising low-doses of cyclophosphamide (CY), pentostatin (PT), and anti-thymocyte globulin (ATG) to the recipient, transplanting a therapeutically effective amount of PD-L1⁺ CD4⁺ T-depleted donor bone marrow cells into the recipient; and optionally transplanting an organ into the recipient. The PD-L1⁺ CD4⁺ T-depleted donor bone marrow cells include donor CD4⁺ T-depleted spleen cells and donor CD4⁺ T-depleted bone marrow cells, and in some embodiments, expression of PD-L1 on the PD-L1⁺ CD4⁺ T-depleted donor bone marrow cells is determined prior to transplantation of the donor bone marrow cells. The PD-L1⁺ CD4⁺ T-depleted donor bone marrow cells may be enriched or selected for PD-L1 expression. Various steps of the methods for promoting or inducing organ transplant tolerance of the present technology may be performed in any order. For example, the recipient may receive an organ transplant prior to or after either of the condition regimen or transplant of the PD-L1⁺ CD4⁺ T-depleted donor bone marrow cells, or the recipient may be administered the conditioning regimen before transplantation of the PD-L1⁺ CD4⁺ T-depleted donor bone marrow cells.

In some embodiments, components of the conditioning regimen such as CY, PT, and ATG are administered individually or in combination to condition a recipient in preparation for and prior to transplantation of donor bone marrow cells.

In some embodiments, the PD-L1⁺ CD4⁺ T-depleted donor bone marrow cells may be haploidentical, haplo-mismatched, full HLA- or MHC-matched, partially HLA- or MHC-matched, HLA- or MHC-mismatched to the recipient. Recent studies indicate that induction of MHC-mismatched mixed chimerism may play an important role in the therapy of autoimmune diseases and conditions as well as in organ transplantation immune tolerance. Thus, according to some embodiments, an HLA- or MHC-mismatched or haploidentical donor may be desirable to avoid disease susceptible loci.

In some embodiments, a population of donor-derived PD-L1⁺ CD8⁺ dendritic cells and/or a population of recipient peripheral T regulatory cells is present in the recipient after organ transplant tolerance has been established.

According to the methods of the present technology, donor organs may be obtained from living donor or a deceased donor. Such organs may be solid organs, such as solid organs selected from the group consisting of heart, lung, liver, kidney, intestine, pancreas, and eye. In some embodiments, the donor organ is skin.

The present technology also includes transplant compositions for transplanting into a recipient. Such transplant compositions may comprise a therapeutically effective amount of PD-L1⁺ CD4⁺ T-depleted donor bone marrow cells and a donor organ, each of which may be administered as separate compositions.

Unless defined otherwise herein, all technical and scientific terms used herein have the same meaning as commonly understood by those of ordinary skill in the art to which the present technology belongs.

The articles “a” and “an” are used herein to refer to one or to more than one (i.e., to at least one) of the grammatical object of the article. By way of example, “an element” means one element or more than one element. Likewise, any reference to singular includes plural embodiments, and any reference to more than one component may include a singular embodiment.

The term “about” means a quantity, level, value, number, frequency, percentage, dimension, size, amount, weight or length that varies by acceptable levels in the art. In some embodiments, such variation may be as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth.

The term “recipient” or “host” as used herein refers to a recipient of transplanted or grafted tissue or cells. These terms may refer to, for example, a recipient of an administration of donor bone marrow, donor T cells, or a tissue graft. The transplanted tissue may be derived from a syngeneic or allogeneic donor. The recipient, host, or subject may be an animal, a mammal, or a human.

The term “donor” as used herein refers to a subject from whom tissue or cells are obtained to be transplanted or grafted into a recipient or host. For example, a donor may be a recipient from whom bone marrow, T cells, or other tissue to be administered to a recipient or host is derived. The donor or recipient may be an animal, a mammal, or a human. In some embodiments, the donor may be an MHC- or HLA-matched donor, meaning the donor shares the same MHC- or HLA with the recipient. In some embodiments, the donor may be MHC- or HLA-mismatched to the recipient.

The term “chimerism” as used herein refers to a state in which one or more cells from a donor are present and functioning in a recipient or host. Recipient tissue exhibiting “chimerism” may contain donor cells only (complete chimerism), or it may contain both donor and host cells (mixed chimerism). “Chimerism” as used herein may refer to either transient or stable chimerism. In some embodiments, the mixed chimerism may be MHC- or HLA-matched mixed chimerism. In some embodiments, the mixed chimerism may be MHC- or HLA-mismatched mixed chimerism.

The term “organ” as used herein refers to a group of cells which perform the same function, a tissue, a graft, or an organoid. The term “solid organ” as used herein refers to a collection of tissues which perform a similar function. A solid organ may be a heart, lung, liver, kidney, intestine, pancreas, eye, or skin.

The terms “transplant tolerance,” “immune tolerance,” or “tolerogenic” are used to describe a state in which the immune system is unresponsive to a particular antigen. In some embodiments, the antigen is from a donor. In some embodiments, the antigen is a self-antigen. In some embodiments, the antigen is on a transplant organ, tissue, organoid, or cell. In some embodiments, immune tolerance prevents inflammatory reactions.

The term “mixed chimerism” refers to a recipient possessing both donor antigens and recipient antigens following transplantation.

The term “conditioning-induced” refers to the MHC-mismatched MC state which appears as a result of treatment with the COH conditioning regimen, or any variation of the conditioning regimens of the present technology.

The term “COH regimen” or “COH conditioning regimen” as used herein refer to the induction of conditioning-induced MCs through the coupling of conditioning and HCT. COH regimen is administered before, after, or during a transplant.

The term “wildtype” as used herein refers to a genetic background in which the gene of interest is not manipulated, whether be by deletion, insertion, substitution, or any combination thereof. Other genes which are not the gene of interest may be subject to genetic manipulation or variation.

The term “anergic” as used herein refers to the state of tolerance in which a cell is active in the periphery, but does not respond to a given stimuli, such as an antigen.

The term “exhausted” as used herein refers to the state of a cell wherein chronic stimuli perturbation or exposure, such as immune stimulation, reduces cell response to stimuli, such as an antigen.

The terms “treat,” “treating,” or “treatment” as used herein with regards to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “recipient,” “subject,” or “subject” as used herein refers to a male or female human, dogs, and animals in models used for clinical research. In some embodiments, the recipient of these methods and compositions is a human receiving a transplant. In further embodiments, the human recipient of these methods and compositions is a prenatal, a newborn, an infant, a toddler, a preschool-aged child, a grade-school-aged child, a teen, a young adult, or an adult. In some embodiments, the recipient is prone to GVHD. In some embodiments, the recipient has GVHD. In some embodiments, the recipient has transplant rejection. In some embodiments, the transplant is an organ, tissue, organoid, or cell transplant. In some embodiments, the transplant is allogenic, autogenic, or xenogeneic.

As used herein, “administering,” “administer,” and “administration” refer to delivery of therapies or compositions of the present technology to a recipient either by local or systemic administration. Administration may intratracheal, intranasal, epidermal and transdermal, oral, or parenteral. Parenteral administration includes intravenous, intraarterial, subcutaneous, intraperitoneal or intramuscular injection or infusion; or intracranial, e.g., intrathecal or intraventricular, administration.

As used herein, the term “a therapeutic level” or “therapeutically effective” means reducing transplant rejection by about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of a control with transplant rejection of a similar transplant type.

The phrase “therapeutically effective amount,” “effective dose,” or “effective amount” as used herein refers to an amount of an agent, population of cells, or composition that produces a desired therapeutic effect. For example, a therapeutically effective amount of donor BM cells or donor CD4⁺ T-depleted spleen cells may refer to that amount that generates chimerism in a recipient. The precise therapeutically effective amount is an amount of the agent, population of cells, or composition that will yield the most effective results in terms of efficacy in a given recipient. This amount will vary depending upon a variety of factors, including but not limited to the characteristics of the therapeutic agent, population of cells, or composition (including activity, pharmacokinetics, pharmacodynamics, and bioavailability), the physiological condition of the recipient (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a recipient's response to administration of an agent, population of cells, or composition and adjusting the dosage accordingly. For additional guidance, see Remington: The Science and Practice of Pharmacy (Gennaro ed. 20th edition, Williams & Wilkins PA, USA) (2005), incorporated herein by reference in its entirety.

In some embodiments, the agents and/or cells administered to a recipient may be part of a pharmaceutical composition. Such a pharmaceutical composition may include one or more of CY, PT and ATG and a pharmaceutically acceptable carrier; or one or more populations of donor cells and a pharmaceutically acceptable carrier. The pharmaceutical compositions of the present technology may include compositions including a single agent or a single type of donor cell (e.g., donor bone marrow cells, donor CD4⁺ T-depleted spleen cells, donor CD8⁺ T cells, or donor G-CSF-mobilized peripheral blood mononuclear cells) in each composition, or alternatively, may include a combination of agents, populations of cells, or both.

A “pharmaceutically acceptable carrier” as used herein refers to a pharmaceutically acceptable material, composition, or vehicle that is involved in carrying or transporting an agent or cell of interest from one tissue, organ, or portion of the body to another tissue, organ, or portion of the body. Such a carrier may comprise, for example, a liquid, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, or some combination thereof. Each component of the carrier is “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Nonlimiting examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) natural polymers such as gelatin, collagen, fibrin, fibrinogen, laminin, decorin, hyaluronan, alginate and chitosan; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as trimethylene carbonate, ethyl oleate and ethyl laurate; (13) agar; (14) buffering agents, such as magnesium hydroxide and aluminum hydroxide; (15) alginic acid (or alginate); (16) pyrogen-free water; (17) isotonic saline; (18) Ringer's solution; (19) alcohol, such as ethyl alcohol and propane alcohol; (20) phosphate buffer solutions; (21) thermoplastics, such as polylactic acid, polyglycolic acid, (22) polyesters, such as polycaprolactone; (23) self-assembling peptides; and (24) other non-toxic compatible substances employed in pharmaceutical formulations such as acetone.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, and the like.

In some embodiments, the pharmaceutically acceptable carrier is an aqueous carrier, e.g., buffered saline and the like. In some embodiments, the pharmaceutically acceptable carrier is a polar solvent, e.g., acetone and alcohol.

The term “low dose” as used herein refers to a dose of a particular agent, such as cyclophosphamide (CY), pentostatin (PT), or anti-thymocyte globulin (ATG), and is lower than a conventional dose of each agent used in a conditioning regimen, particularly in a myeloablative conditioning regimen. For example, the dose may be about 5%, about 10%, about 15%, about 20% or about 30% lower than the standard dose for conditioning. In some embodiments, a low dose of CY may be from about 30 mg/kg to about 75 mg/kg; a low dose of PT is about 1 mg/kg; and a low dose of ATG may be from about 25 mg/kg to about 50 mg/kg. In general, different animals require different doses and human doses are lower than mouse doses.

The term “simultaneously” as used herein with regards to administration of two or more agents means that the agents are administered at the same or nearly the same time. For example, two or more agents are considered to be administered “simultaneously” if they are administered via a single combined administration, two or more administrations occurring at the same time, or two or more administrations occurring in succession without extended intervals in between.

### Conditioning Agents

Allogenic HCT may be used to promote MC for organ transplants. However, transplant techniques utilizing MCs may carry a risk of malignant inflammatory responses or transplant rejection, which may be reduced by specifically utilizing conditioning-induced MCs. Conditioning-induced MCs may promote or increase success of cell, organoid, tissue, or organ transplant tolerance. Transplant tolerance may include immunoablation or reducing or preventing the risk of transplant rejection, inflammatory reactions, or tumor burden. Conditioning-induced MCs are generated using conditioning agents and the conditioning regimens of the present technology. Conditioning agents help promote, select, or enrich specific cell types or molecules to be used in transplantation. Conditioning agents may include an alkylating agent, an antineoplastic agent, an antimetabolite agent, a purine analog, or an antibody. In some embodiments, the conditioning treatment comprises the conditioning agents CY, PT, or ATG.

CY is an alkylating agent whose main effect is due to its metabolite phosphoramide mustard. This metabolite is only formed in cells that have low levels of aldehyde dehydrogenase (ALDH). Phosphoramide mustard creates nucleotide crosslinks between and within DNA strands at guanine N-7 positions, leading to cell apoptosis. Cyclophosphamide has relatively little typical chemotherapy toxicity, as ALDHs only are present in relatively large concentrations in bone marrow, liver and intestinal epithelial cells. Treg cells express higher levels of ALDH than conventional T cells. For these reasons, CY has been used in combination with radiation as part of preconditioning regimens or post-transplantation immunosuppressants in HCT and organ transplantation.

Pentostatin (PT) is a purine analog that may result in lymphocyte toxicity by inhibiting adenosine deaminase. An inherited deficiency of adenosine deaminase causes a disease in which both T and B cells fail to mature. In the setting of hairy cell leukemia or GVHD therapy, pentostatin results in profound reduction of absolute T cell counts and relative increase of myeloid cells; this may reduce the incidence of infection associated with conditioning induced lymphocyte depletion.

Anti-thymocyte globulin (ATG) has been used in combination with total lymphoid irradiation (TLI) as a non-myeloablative conditioning regimen for HCT and induction of mixed chimerism in both animal models and humans.

### Dosing and Administration

Although high-dose conditioning agents (e.g., CY and PT) have been used to condition recipients with hematological malignancies as a preparation for an HLA-matched or haplo-mismatched HCT, recipients may develop complete chimerism and GVHD. TLI and ATG conditioning causes undesirable side effects as well, such as short-term toxicity. Therefore, the dosages and administration of these agents are important in determining safety and transplant tolerance.

In some embodiments, the dose of CY used in the conditioning regimens and methods of the present technology may be from at least about 50 mg to at least about 1000 mg, from at least about 100 mg to at least about 800 mg, from at least about 150 mg to at least about 750 mg, from at least about 200 mg to at least about 500 mg, at least about 100 mg, at least about 200 mg, at least about 300 mg, at least about 400 mg, at least about 500 mg, at least about 600 mg, at least about 700 mg, or at least about 800 mg.

In some embodiments, the dose of ATG used in the conditioning regimens and methods of the present technology may be from at least about 0.5 mg/kg/day to at least about 10 mg/kg/day, from at least about 1.0 mg/kg/day to at least about 8.0 mg/kg/day, from at least about 1.5 mg/kg/day to at least about 7.5 mg/kg/day, from at least about 2.0 mg/kg/day to at least about 5.0 mg/kg/day, at least about 0.5 mg/kg/day, at least about 1.0 mg/kg/day, at least about 1.5 mg/kg/day, at least about 2.0 mg/kg/day, at least about 2.5 mg/kg/day, at least about 3.0 mg/kg/day, at least about 3.5 mg/kg/day, at least about 4.0 mg/kg/day, at least about 4.5 mg/kg/day, or at least about 5.0 mg/kg/day.

In some embodiments, the dose of PT used in the conditioning regimens and methods of the present technology may be from at least about 1 mg/m²/dose to at least about 10 mg/m²/dose, from at least about 2 mg/m²/dose to at least about 8 mg/m²/dose, from at least about 3 mg/m²/dose to at least about 5 mg/m²/dose, at least about 1 mg/m²/dose, at least about 2 mg/m²/dose, at least about 3 mg/m²/dose, at least about 4 mg/m²/dose, at least about 5 mg/m²/dose, at least about 6 mg/m²/dose, at least about 7 mg/m²/dose, at least about 8 mg/m²/dose, at least about 9 mg/m²/dose, or at least about 10 mg/m²/dose.

In some embodiments, the dose of CY used in the conditioning regimens and methods of the present technology may be from about 50 mg to about 1000 mg, from about 100 mg to about 800 mg, from about 150 mg to about 750 mg, from about 200 mg to about 500 mg, about 100 mg, about 200 mg, about 300 mg, about 400 mg, about 500 mg, about 600 mg, about 700 mg, or about 800 mg. In some embodiments, the human dose of ATG used in the conditioning regimens and methods of the present technology may be from about 0.5 mg/kg/day to about 10 mg/kg/day, from about 1.0 mg/kg/day to about 8.0 mg/kg/day, from about 1.5 mg/kg/day to about 7.5 mg/kg/day, from about 2.0 mg/kg/day to about 5.0 mg/kg/day, about 0.5 mg/kg/day, about 1.0 mg/kg/day, about 1.5 mg/kg/day, about 2.0 mg/kg/day, about 2.5 mg/kg/day, about 3.0 mg/kg/day, about 3.5 mg/kg/day, about 4.0 mg/kg/day, about 4.5 mg/kg/day, or about 5.0 mg/kg/day. In some embodiments, the dose of PT used in the conditioning regimens and methods of the present technology may be from about 1 mg/m²/dose to about 10 mg/m²/dose, from about 2 mg/m²/dose to about 8 mg/m²/dose, from about 3 mg/m²/dose to about 5 mg/m²/dose, about 1 mg/m²/dose, about 2 mg/m²/dose, about 3 mg/m²/dose, about 4 mg/m²/dose, about 5 mg/m²/dose, about 6 mg/m²/dose, about 7 mg/m²/dose, about 8 mg/m²/dose, about 9 mg/m²/dose, or about 10 mg/m²/dose.

In some embodiments, the dose of CY used in the conditioning regimens and methods of the present technology may be from at least about 50 mg to at least 1000 mg, from at least 100 mg to at least 800 mg, from at least 150 mg to at least 750 mg, from at least 200 mg to at least 500 mg, at least 100 mg, at least 200 mg, at least 300 mg, at least 400 mg, at least 500 mg, at least 600 mg, at least 700 mg, or at least 800 mg. In some embodiments, the human dose of ATG used in the conditioning regimens and methods of the present technology may be from at least 0.5 mg/kg/day to at least 10 mg/kg/day, from at least 1.0 mg/kg/day to at least 8.0 mg/kg/day, from at least 1.5 mg/kg/day to at least 7.5 mg/kg/day, from at least 2.0 mg/kg/day to at least 5.0 mg/kg/day, at least 0.5 mg/kg/day, at least 1.0 mg/kg/day, at least 1.5 mg/kg/day, at least 2.0 mg/kg/day, at least 2.5 mg/kg/day, at least 3.0 mg/kg/day, at least 3.5 mg/kg/day, at least 4.0 mg/kg/day, at least 4.5 mg/kg/day, or at least 5.0 mg/kg/day. In some embodiments, the dose of PT used in the conditioning regimens and methods of the present technology may be from at least 1 mg/m²/dose to at least 10 mg/m²/dose, from at least about 2 mg/m²/dose to at least 8 mg/m²/dose, from at least 3 mg/m²/dose to at least 5 mg/m²/dose, at least 1 mg/m²/dose, at least 2 mg/m²/dose, at least 3 mg/m²/dose, at least 4 mg/m²/dose, at least 5 mg/m²/dose, at least 6 mg/m²/dose, at least 7 mg/m²/dose, at least 8 mg/m²/dose, at least 9 mg/m²/dose, or at least 10 mg/m²/dose.

In some embodiments, the conditioning regimens and methods of the present technology include administering the CY, PT, and/or ATG on a daily, weekly, or other regular schedule. For example, administration of CY may be daily; administration of PT may be weekly or at an interval greater than every day (e.g., every two, every three, or every four days); and administration of ATG may be daily, weekly, or at an interval greater than every day (e.g., every two or three days).

In some embodiments, a dose of CY may be administered to the recipient on a daily basis for up to about 28 days, up to about 21 days, up to about 14 days, up to about 12 days, or up to about 7 days prior to transplantation. In some embodiments, a dose of CY may be administered to the recipient every other day for up to about 28 days, up to about 21 days, up to about 14 days, or up to about 7 days prior to transplantation. In one example, a dose of CY may be administered to the recipient on a daily basis for about 21 days prior to transplantation.

In some embodiments, a dose of PT may be administered to the recipient every day, every other day, every third day, every fourth day, every fifth day, every sixth day, or every week for up to about 28 days, up to about 21 days, up to about 14 days, up to about 12 days or up to about 7 days prior to transplantation. In some embodiments, a dose of PT may be administered to the recipient every week for about 21 days prior to transplantation. In some embodiments, a dose of PT may be administered to the recipient every two, three, or four days starting about 3 weeks prior to transplantation. In some embodiments, 3 doses of PT may be administered to the recipient for a week starting about 3 weeks prior to transplantation.

In some embodiments, a dose of ATG may be administered to the recipient every other day, every third day, every fourth day or every fifth day for up to about 28 days, up to about 21 days, up to about 14 days, up to about 12 days or up to about 7 days prior to transplantation. For example, a dose of ATG may be administered to the recipient every third day for about 21 days prior to transplantation. In some embodiments, a dose of ATG may be administered for two, three, or four days in a row about 7 days, 8 days, 9 days, 10 days, 11 days, 12 days, 13 days, or 14 days prior to transplantation. In some embodiments, a dose of ATG may be administered for 5 days in a row starting about two weeks prior to transplantation.

In some embodiments, the recipient is administered at least about 1 dose, at least about 2 doses, at least about 3 doses, at least about 3 doses, at least about 4 doses, at least about 5 doses, at least about 6 doses, at least about 7 doses, at least about 8 doses, at least about 9 doses, at least about 10 doses, at least about 12 doses, at least about 14 doses, at least about 16 doses, at least about 18 doses, at least about 20 doses, or more, of the CY, the ATG, and/or the PT.

In some embodiments, the conditioning regimen includes (i) three doses of PT at a dose of about 4 mg/m²/dose may be administered to a human recipient about 3 weeks, about 2 weeks and about 1 week before transplantation; (ii) three, four, or five doses of ATG at a dose of about 1.5 mg/kg/day may be administered to a human recipient about 12 days, about 11 days, and about 10 days before transplantation; and (iii) CY at a dose of about 200 mg orally may be administered to a human recipient on a daily basis about 3 weeks before transplantation.

The conditioning regimen may comprise administering one or more doses of a conditioning agent (e.g., CY, PT, and/or ATG) to the recipient.

The recipient may be administered one or more doses of a single conditioning agent. In some embodiments, the recipient is administered one or more doses of CY and is not administered a dose of PT, and/or ATG. In some embodiments, the recipient is administered one or more doses of PT and is not administered a dose of CY, and/or ATG. In some embodiments, the recipient is administered one or more doses of ATG and is not administered a dose of PT, and/or CY.

The recipient may be administered one or more doses of two different conditioning agents. In some embodiments, administered one or more doses of CY and one or more doses of PT and is not administered a dose of ATG. In some embodiments, the recipient is administered one or more doses of PT and one or more doses of ATG and is not administered a dose of CY. In some embodiments, the recipient is administered one or more doses of ATG and one or more doses of CY and is not administered a dose of PT.

The recipient may be administered one or more doses of three different conditioning agents. In some embodiments, the recipient is administered one or more doses of CY, one or more doses of PT, and one or more doses of ATG.

The one or more doses of the conditioning agents (e.g., CY, PT, and/or ATG) may be administered with an immunosuppressant.

In some embodiments, when the recipient is administered at least two different conditioning agents (e.g., two or more different conditioning agents or three or more different conditioning agents), the at least two different conditioning agents may be administered simultaneously (e.g., CY and PT administered simultaneously; CY and ATG administered simultaneously; PT and ATG administered simultaneously; or CY, PT, and ATG are administered simultaneously).

In some embodiments, when the recipient is administered at least two different conditioning agents, the at least two different conditioning agents may be administered sequentially (i.e., “consecutively”) (e.g., CY and PT administered sequentially; CY and ATG administered sequentially; PT and ATG administered sequentially; or CY, PT, and ATG are administered sequentially).

In some embodiments, the recipient is administered 7.5-13.0×10\hat{ }6 cells/kg of recipient body weight (BW) of CD34⁺ cells. In some embodiments, CY is administered at 200 mg/day for 19 days. In some embodiments, CY is administered at 60 mg/kg/day. In some embodiments, the administration of CY is on Day −22 to Day −4. In some embodiments, CY is administered on Day −3 and Day −2. In some embodiments, CY is administered orally. In some embodiments, CY is administered intravenously. In some embodiments, PT is administered at 4 mg/m²/dose. In some embodiments, the PT administration of PT is on Days −22, −19, −15, and −12. In some embodiments, PT is administered intravenously. In some embodiments, ATG is administered at 1.5 mg/kg/dose. In some embodiments, ATG is administered on Days −13 to −9. In some embodiments, ATG is administered intravenously. In some embodiments, ATG is rabbit ATG.

In some embodiments, the recipient is administered greater than or equal to 2×106 or 5×106⁻CD34⁺ cells/kg of recipient BW. In some embodiments, the recipient is administered greater than or equal to 10×106⁻CD34⁺ cells/kg of recipient BW. In some embodiments, depletion of CD4⁺ T cells is greater than 97% of the CD4⁺ T cells. In some embodiments, CY is administered at 200 mg/day for 19 days. In some embodiments, CY is administered at 60 mg/kg/day. In some embodiments, the administration of CY is on Day −22 to Day −4. In some embodiments, CY is administered on Day −3 and Day −2. In some embodiments, CY is administered orally. In some embodiments, CY is administered intravenously. In some embodiments, PT is administered at 4 mg/m²/dose. In some embodiments, the PT administration of PT is on Days −22, −19, −15, and −12. In some embodiments, PT is administered intravenously. In some embodiments, ATG is administered at 1.5 mg/kg/dose. In some embodiments, ATG is administered on Days −13 to −9. In some embodiments, ATG is administered intravenously. In some aspects, ATG is rabbit ATG. In some embodiments, a 200 mg/day administration of CY for 21 days is administered to the recipient in combination with PT. In some embodiments, the administration of PT is 4 mg/m² provided in 4 daily doses. In some embodiments, the administration of ATG is five daily doses of 1.5 mg/kg. In some embodiments, the ATG is rabbit ATG.

It is within the purview of one of ordinary skill in the art to select a suitable route of administration of the conditioning agents of the present technology (e.g., CY, PT, and ATG). For example, these conditioning agents may be administered by oral administration including sublingual and buccal administration, and parenteral administration including intravenous administration, intramuscular administration, and subcutaneous administration. Different doses of the same conditioning agent may be administered by different routes. When at least two or more different conditioning agents are administered to the recipient, the different conditioning agents may be administered by the same route or by different routes (e.g., one or more of CY, PT, and ATG are each administered intravenously; CY is administered orally and ATG and PT are administered intravenously).

### Cell Selection and Depletion

Donor and/or recipient cells may be selected, enriched, or depleted to enhance effects of the conditioning agents of the present technology. For example, the depletion of donor-derived CD4⁺ T cells and detection, enrichment, and/or selection of PD-L1⁺ cells from a population of bone marrow cells and/or the conditioning agents of the present technology (e.g., the combination of CY, PT and ATG) allows lowering the dose of each of the conditioning agents, thereby to reduce the toxic side effects while achieving mixed chimerism. It is within the purview of one of ordinary skill in the art to deplete CD4⁺ T cells and detect, enrich, and/or select for PD-L1⁺ cells from a population of bone marrow cells. It is also within the purview of one of ordinary skill in the art to adjust the dose of each conditioning agent (e.g., CY, PT, and ATG) to achieve the desired effect.

In some embodiments, CD4⁺ T cell depletion comprises at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% of the CD4⁺ T cells.

In some embodiments, PD-L1⁺ cell selection comprises at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% of the PD-L1⁺ cells.

### Mixed Chimerism

Mixed chimerism may be induced by conditioning with the combination of CY, PT, and ATG and supplying to the recipient PD-L1⁺ donor-derived CD4⁺ depleted bone marrow cells that facilitate engraftment. In some embodiments, the methods of the present technology may include transplantation of PD-L1⁺ donor-derived CD4⁺ T-depleted bone marrow cells before or following administration of one or more conditioning agents (e.g., CY, PT, and/or ATG) in accordance with the conditioning regimens of the present technology. In some embodiments, the methods in accordance with the present technology may include administering PD-L1⁺ donor-derived CD4⁺ T-depleted bone marrow cells before or following administration of a conditioning agent (e.g., CY, PT, and/or ATG).

The present technology may comprise methods of inducing stable mixed chimerism in a recipient by administration of radiation-free, low doses of conditioning agents (e.g., CY, PT, and/or ATG), followed by transplantation of PD-L1⁺ donor-derived CD4⁺ T-depleted bone marrow cells. In some embodiments, mixed chimerism in a recipient is induced by administration of radiation-free, low doses of CY, PT, and ATG and a therapeutically effective amount of PD-L1⁺ donor-derived CD4⁺ T-depleted bone marrow cells. CY, PT, and ATG are administered to the recipient before or after transplantation, in accordance with the conditioning regimen described above.

The donor cells may be MHC- or HLA-matched or MHC- or HLA-mismatched. In some embodiments, the mixed chimerism is HLA- or MHC-mismatched mixed chimerism. In some embodiments, stable mixed chimerism is haploidentical stable mixed chimerism.

In some embodiments, the donor organ and/or the donor cells are haploidentical to the recipient and in some embodiments, the donor organ and/or the donor cells are haplo-mismatched to the recipient. In some embodiments, the donor organ and/or the donor cells are not full-HLA- or MHC-matched to the recipient.

### Associated Methods

### Increasing Transplant Success

The conditioning regimens (e.g., conditioning agents, dosing, and administration) of the present technology may be used in transplant-related contexts. This may include use of the conditioning regimens to increase success (e.g., induce tolerance or reduce rejection) of a transplant, treat transplant rejection, or treat or prevent graft-versus-host disease (GVHD) in a subject (i.e., “recipient”) in need thereof, relative to a control.

In some embodiments, the conditioning regimens of the present technology are used to increase success of transplantation of an organ in a recipient. In some embodiments, conditioning is administered before transplantation of the organ. In some embodiments, conditioning is administered at least about one minute, at least about one hour, at least about one day, at least about one week, at least about two weeks, at least about one month, at least about two months, at least about six months, or at least about one year before transplantation of the organ. In some embodiments, conditioning is administered after transplantation of the organ. In some embodiments, conditioning is administered at least about one minute, at least about one hour, at least about one day, at least about one week, at least about two weeks, at least about one month, at least about two months, at least about six months, at least about or one year after transplantation of the organ. Conditioning may be administered during transplantation of the organ. In some embodiments, the organ is a solid organ. Nonlimiting examples of a solid organ include a heart, a lung, a liver, a kidney, an intestine, a pancreas, or an eye. In some embodiments, the solid organ is skin.

In some embodiments, conditioning is used to prevent transplant rejection. In some embodiments, conditioning is used to treat transplant rejection. In some embodiments, conditioning is used to reduce transplant rejection. In some embodiments, conditioning is used to prevent GVHD. In some embodiments, conditioning is used to treat GVHD. In some embodiments, conditioning is used to reduce GVHD. In some embodiments, conditioning is used to prevent proinflammatory reactions. In some embodiments, conditioning is used to treat outcomes of proinflammatory reactions. In some embodiments, conditioning is used to reduce proinflammatory reactions. In some embodiments, conditioning is used to promote immunoablation.

In some embodiments, conditioning is radiation free. In some embodiments, conditioning is non-myeloablative.

HCT may be supplemented with conditioning regimens to better promote or induce transplant tolerance, where host-type bone marrow or spleen cells may be transplanted into a recipient. Additionally, enriching for, selecting for, or isolating hematopoietic cells which express PD-L1 or PD-1 may be important in transplant tolerance. PD-L1-expressing donor-type tolerogenic DCs (i.e., CD8⁺ DC) may induce residual donor-reactive host-type T cells into anergy or exhaustion status and induce their differentiation into pTreg cells, and the pTreg cells may turn donor-type DCs carrying tissue-specific antigens from the organ transplant into tolerogenic DCs, such that long-term organ transplant tolerance is established and maintained. In some embodiments, the present technology comprises a means for promoting or inducing transplant tolerance by enriching or selecting for specific molecules or cells for HCT.

In some embodiments, a therapeutically effective amount of PD-L1⁺ donor-derived CD4⁺ T-depleted bone marrow cells are transplanted into a recipient. In some embodiments, a therapeutically effective amount of donor-derived CD4⁺ depleted bone marrow cells are transplanted into a recipient. In some embodiments, a therapeutically effective amount of PD-L1⁺ donor-derived CD4⁺ T-depleted spleen cells are transplanted into a recipient. In some embodiments, a therapeutically effective amount of donor-derived CD4⁺ depleted spleen marrow cells are transplanted into a recipient. In some embodiments, a therapeutically effective amount of PD-L1⁺ donor-derived CD4⁺ T-depleted bone marrow and spleen cells are transplanted into a recipient. In some embodiments, a therapeutically effective amount of donor-derived CD4⁺ depleted bone marrow and spleen marrow cells are transplanted into a recipient. In some embodiments, the PD-L1⁺ donor-derived CD4⁺ T-depleted bone marrow or spleen cells are transplanted into a recipient before, during, or after transplantation of an organ in the recipient. In some embodiments, the PD-L1⁺ donor-derived CD4⁺ T-depleted bone marrow or spleen cells are conditioned with a regimen comprising low-doses of CY, PT, and ATG to the recipient. In some embodiments, the PD-L1⁺ donor-derived CD4⁺ T-depleted bone marrow cells include donor-derived CD4⁺ T-depleted spleen cells, and donor-derived CD4⁺ T-depleted bone marrow cells. In some embodiments, donor bone marrow cells are selected for prior to the transplantation of a therapeutically effective amount of PD-L1⁺ donor-derived CD4⁺ T-depleted bone marrow cells into the recipient. In some embodiments, donor-derived bone marrow cells are enriched for prior to the transplantation of a therapeutically effective amount of PD-L1⁺ donor-derived CD4⁺ T-depleted bone marrow cells into the recipient. In some embodiments, a population of PD-L1⁺ cells is isolated from the donor-derived CD4⁺ T-depleted bone marrow cells.

In some embodiments, donor bone marrow cells are selected for prior to transplantation of an organ in a recipient. In some embodiments, donor bone marrow cells are enriched for prior to transplantation of an organ in a recipient. In some embodiments, donor bone marrow cells are selected for or enriched for at least one minute, one hour, one day, one week, one month, or one year prior to transplantation of an organ in a recipient. In some embodiments, the conditioning enriches for or selects PD-L1⁺ donor-derived CD4⁺ T-depleted bone marrow cells. In some embodiments, the conditioning enriches for or selects donor-derived CD4⁺ T-depleted bone marrow cells. In some embodiments, a population of PD-L1⁺ cells is isolated from the donor-derived CD4⁺ T-depleted bone marrow cells. In some embodiments, the PD-L1⁺ donor-derived CD4⁺ T-depleted bone marrow cells include donor-derived CD4⁺ T-depleted spleen cells, and donor-derived CD4⁺ T-depleted bone marrow cells. In some embodiments, expression of PD-L1 on the PD-L1⁺ donor-derived CD4⁺ T-depleted bone marrow cells is determined prior to transplantation of an organ in a recipient. In some embodiments, the PD-L1⁺ donor-derived CD4⁺ T-depleted bone marrow cells are selected based on PD-L1⁺ expression prior to transplantation of an organ in a recipient. In some embodiments, the PD-L1⁺ donor-derived CD4⁺ T-depleted bone marrow cells are enriched for PD-L1⁺ expression prior to transplantation of an organ in a recipient. In some embodiments, the organ is a solid organ. In some embodiments, the solid organ is a heart, a lung, a liver, a kidney, an intestine, a pancreas or an eye. In some embodiments, the solid organ is skin.

In some embodiments, donor bone marrow cells are selected for after transplantation of an organ in a recipient. In some embodiments, donor bone marrow cells are enriched for after transplantation of an organ in a recipient. In some embodiments, donor bone marrow cells are selected for or enriched for at least one minute, one hour, one day, one week, one month, or one year after transplantation of an organ in a recipient. In some embodiments, the conditioning enriches for or selects for PD-L1⁺ bone marrow cells which are also CD4⁺ T-depleted. In some embodiments, the conditioning enriches for or selects donor-derived CD4⁺ T-depleted bone marrow cells. In some embodiments, a population of PD-L1⁺ cells is isolated from the CD4⁺ T-depleted donor bone marrow cells. In some embodiments, the PD-L1⁺ and CD4⁺ T-depleted bone marrow cells include donor CD4⁺ T-depleted spleen cells, and donor CD4⁺ T-depleted bone marrow cells. In some embodiments, expression of PD-L1 on the bone marrow cells, such as the CD4⁺ T-depleted donor bone marrow cells and/or the PD-L1⁺ and CD4⁺ T-depleted bone marrow cells is determined before or after transplantation of an organ in a recipient. In some embodiments, the CD4⁺ T-depleted PD-L1⁺ donor bone marrow cells are selected based on PD-L1⁺ expression before or after transplantation of an organ in a recipient. In some embodiments, the CD4⁺ T-depleted PD-L1⁺ donor bone marrow cells are enriched for PD-L1⁺ expression before or after transplantation of an organ in a recipient. In some embodiments, the organ is a solid organ. In some embodiments, the solid organ is a heart, a lung, a liver, a kidney, an intestine, a pancreas or an eye. In some embodiments, the solid organ is skin.

In some embodiments, donor bone marrow cells are selected for during transplantation of an organ in a recipient. In some embodiments, donor bone marrow cells are enriched for during transplantation of an organ in a recipient. In some embodiments, the conditioning enriches for or selects PD-L1⁺ donor-derived CD4⁺ T-depleted bone marrow cells. In some embodiments, the conditioning enriches for or selects donor-derived CD4⁺ T-depleted bone marrow cells. In some embodiments, a population of PD-L1⁺ cells is isolated from the donor-derived CD4⁺ T-depleted bone marrow cells. In some embodiments, the PD-L1⁺ donor-derived CD4⁺ T-depleted bone marrow cells include donor-derived CD4⁺ T-depleted spleen cells, and donor-derived CD4⁺ T-depleted bone marrow cells. In some embodiments, expression of PD-L1 on the PD-L1⁺ donor-derived CD4⁺ T-depleted bone marrow cells is determined during transplantation of an organ in a recipient. In some embodiments, the PD-L1⁺ donor-derived CD4⁺ T-depleted bone marrow cells are selected based on PD-L1⁺ expression during transplantation of an organ in a recipient. In some embodiments, the PD-L1⁺ donor-derived CD4⁺ T-depleted bone marrow cells are enriched for PD-L1⁺ expression during transplantation of an organ in a recipient. In some embodiments, the organ is a solid organ. In some embodiments, the solid organ is a heart, a lung, a liver, a kidney, an intestine, a pancreas or an eye. In some embodiments, the solid organ is skin.

In some embodiments, donor spleen cells are selected for prior to transplantation of an organ in a recipient. In some embodiments, donor spleen cells are enriched for prior to transplantation of an organ in a recipient. In some embodiments, donor spleen cells are selected for or enriched for at least one minute, one hour, one day, one week, one month, or one year prior to transplantation of an organ in a recipient. In some embodiments, the conditioning enriches for or selects PD-L1⁺ donor-derived CD4⁺ T-depleted spleen cells. In some embodiments, a population of PD-L1⁺ cells is isolated from the donor-derived CD4⁺ T-depleted spleen cells. In some embodiments, the conditioning enriches for or selects donor-derived CD4⁺ T-depleted spleen marrow cells. In some embodiments, the PD-L1⁺ donor-derived CD4⁺ T-depleted spleen cells include donor-derived CD4⁺ T-depleted spleen cells, and donor-derived CD4⁺ T-depleted bone marrow cells. In some embodiments, expression of PD-L1 on the PD-L1⁺ donor-derived CD4⁺ T-depleted spleen cells is determined prior to transplantation of an organ in a recipient. In some embodiments, the PD-L1⁺ donor-derived CD4⁺ T-depleted spleen cells are selected based on PD-L1⁺ expression prior to transplantation of an organ in a recipient. In some embodiments, the PD-L1⁺ donor-derived CD4⁺ T-depleted spleen cells are enriched for PD-L1⁺ expression prior to transplantation of an organ in a recipient. In some embodiments, the organ is a solid organ. In some embodiments, the solid organ is a heart, a lung, a liver, a kidney, an intestine, a pancreas or an eye. In some embodiments, the solid organ is skin.

In some embodiments, donor spleen cells are selected for after transplantation of an organ in a recipient. In some embodiments, donor spleen cells are enriched for after transplantation of an organ in a recipient. In some embodiments, donor spleen cells are selected for or enriched for at least one minute, one hour, one day, one week, one month, or one year after transplantation of an organ in a recipient. In some embodiments, the conditioning enriches for or selects PD-L1⁺ donor-derived CD4⁺ T-depleted spleen cells. In some embodiments, the conditioning enriches for or selects donor-derived CD4⁺ T-depleted spleen marrow cells. In some embodiments, a population of PD-L1⁺ cells is isolated from the donor-derived CD4⁺ T-depleted spleen cells. In some embodiments, the PD-L1⁺ CD4⁺ T-depleted donor spleen cells include donor-derived CD4⁺ T-depleted spleen cells, and donor-derived CD4⁺ T-depleted bone marrow cells. In some embodiments, expression of PD-L1 on the PD-L1⁺ donor-derived CD4⁺ T-depleted spleen cells is determined after transplantation of an organ in a recipient. In some embodiments, the PD-L1⁺ donor-derived CD4⁺ T-depleted spleen cells are selected based on PD-L1⁺ expression after transplantation of an organ in a recipient. In some embodiments, the PD-L1⁺ donor-derived CD4⁺ T-depleted spleen cells are enriched for PD-L1⁺ expression after transplantation of an organ in a recipient. In some embodiments, the organ is a solid organ. In some embodiments, the solid organ is a heart, a lung, a liver, a kidney, an intestine, a pancreas or an eye. In some embodiments, the solid organ is skin.

In some embodiments, donor spleen cells are selected for during transplantation of an organ in a recipient. In some embodiments, donor spleen cells are enriched for during transplantation of an organ in a recipient. In some embodiments, the conditioning enriches for or selects PD-L1⁺ donor-derived CD4⁺ T-depleted spleen cells. In some embodiments, the conditioning enriches for or selects donor-derived CD4⁺ T-depleted spleen cells. In some embodiments, a population of PD-L1⁺ cells is isolated from the donor-derived CD4⁺ T-depleted spleen cells. In some embodiments, the PD-L1⁺ donor-derived CD4⁺ T-depleted spleen cells include donor-derived CD4⁺ T-depleted spleen cells, and donor-derived CD4⁺ T-depleted bone marrow cells. In some embodiments, expression of PD-L1 on the PD-L1⁺ donor-derived CD4⁺ T-depleted spleen cells is determined during transplantation of an organ in a recipient. In some embodiments, the PD-L1⁺ donor-derived CD4⁺ T-depleted spleen cells are selected based on PD-L1⁺ expression during transplantation of an organ in a recipient. In some embodiments, the PD-L1⁺ donor-derived CD4⁺ T-depleted spleen cells are enriched for PD-L1⁺ expression during transplantation of an organ in a recipient. In some embodiments, the organ is a solid organ. In some embodiments, the solid organ is a heart, a lung, a liver, a kidney, an intestine, a pancreas or an eye. In some embodiments, the solid organ is skin.

In some embodiments a population of donor-derived PD-L1⁺ CD8⁺ dendritic cells is present in the recipient after organ transplant tolerance has been established. In some embodiments a population of donor-derived PD-L1⁺ CD8⁺ dendritic cells is present in the recipient during establishment of organ transplant tolerance.

In some embodiments a population of PD-1⁺ T cells is present in the recipient before transplantation of an organ in a recipient. In some embodiments a population of PD-1⁺ T cells is present in the recipient after transplantation of an organ in a recipient. In some embodiments a population of PD-1⁺ T cells is present in the recipient during transplantation of an organ in a recipient.

In some embodiments, PD-L1 expression in the donor cells is measured in a population of CD4⁺ T-depleted donor bone marrow cells. In some embodiments, a population of PD-L1⁺ donor-derived CD4⁺ T-depleted bone marrow cells is selected from the population of CD4⁺ T-depleted bone marrow cells. In some embodiments, selecting comprises enriching for the population of PD-L1⁺ cells from the donor-derived CD4⁺ T-depleted bone marrow cells, and optionally isolating the population of PD-L1⁺ cells from the donor-derived CD4⁺ T-depleted bone marrow cells.

In some embodiments, population of recipient peripheral T regulatory cells is present in the recipient after organ transplant tolerance has been established. In some embodiments, population of recipient peripheral T regulatory cells is present in the recipient during establishment of organ transplant tolerance. In some embodiments, the population of donor PD-L1⁻CD8⁺ dendritic cells is derived from the transplanted bone marrow cells. In some embodiments, the population of recipient peripheral T regulatory cells expands in the recipient after organ transplant tolerance has been established.

In some embodiments, the recipient has been conditioned with a regimen comprising low-doses of CY, PT, and ATG.

In some embodiments, a population of conditioning cells that facilitate engraftment during HCT is administered to the recipient. In some embodiments, the population of conditioning cells that facilitate engraftment during HCT is selected from one or more populations of conditioning donor cells selected from CD4⁺ T-depleted spleen cells, CD8⁺ T cells, and Granulocyte colony-stimulating factor (G-CSF)-mobilized peripheral blood mononuclear cells. In some embodiments, transplantation of the population of donor bone marrow cells occurs on the same day as or after the administration of the population of conditioning cells that facilitate engraftment during HCT. In some embodiments, the population of conditioning donor cells, the population of donor bone marrow cells, or both are MHC- or HLA-mismatched to the recipient. In some embodiments, the population of conditioning cells that facilitate engraftment during HCT is derived from the same donor as the CD4⁺ T-depleted PD-L1⁺ bone marrow cells. In some embodiments, the population of conditioning cells that facilitate engraftment during HCT and the CD4⁺ T-depleted PD-L1⁺ bone marrow cells are derived from different donors. In some embodiments, the organ transplant donor is the same as at least one or as both of the donor from which the population of conditioning cells that facilitate engraftment during HCT are derived and the donor from which the CD4⁺ T-depleted PD-L1⁺ bone marrow cells are derived. In some embodiments, the organ transplant donor is different from at least one or as both of the donor from which the population of conditioning cells that facilitate engraftment during HCT are derived and the donor from which the CD4⁺ T-depleted PD-L1⁺ bone marrow cells are derived.

### Donors

Donor features may impact transplant tolerance, where molecules present on donor cells or organs, such as cell surface ligands or receptors, are recognized by the host immune system. Induction of MCs may permit coexistence of donor and recipient cells following transplant without proinflammatory reaction. Previously, it was thought that donor-type DC expression of MHCII is required for tolerance of MS, and that hematopoietic transplants are not able to induce stable MCs. Conditioning-induced MCs under the COH regimen described herein may to evade the need for donor-type DC expression of MHCII while also permitting the use of different host types with greater transplant tolerance.

In some embodiments, conditioning-induced MCs under the COH regimen comprise a living donor. In some embodiments, the donor is deceased. In some embodiments, the donor is mammalian. In some embodiments, the donor is human. In some embodiments, a donor tissue is sourced from a solid organ. In some embodiments, the donor tissue comprises heart, lung, liver, kidney, intestine, pancreas, or eye tissue, In some embodiments, the donor tissue comprises skin tissue.

In some embodiments, conditioning-induced MCs under the COH regimen comprise donor-type DCs which do not express MHCII. In some embodiments, the donor-type DCs express MHCII. In some embodiments, the donor-type DCs express PD-L1. In some embodiments, the donor-type DCs expressing PD-L1 permit expansion of host-type pTregs. In some embodiments, the donor-type DCs are CD8⁺ DCs.

In some embodiments, the donor is haploidentical to the recipient. In some embodiments, the donor is haplomismatched to the recipient. In some embodiments, the donor is not full-HLA or MHC-matched to the recipient.

### Transplant Compositions

The induction or promotion of transplant tolerance described herein may be useful in the generation of transplant compositions. The transplant composition may be transplanted into a recipient to increase likelihood of tolerance or to evade immune rejection. The transplant composition may also be used to generate conditioning-induced MCs. In some embodiments, the transplant composition comprises a therapeutically effective amount of PD-L1⁺ donor-derived CD4⁺ T-depleted bone marrow cells. In some embodiments, the transplant composition comprises a donor organ. In some embodiments, the recipient has been conditioned with a regimen comprising low-doses of CY, PT, and ATG to the recipient. In some embodiments, the transplant composition comprises a first composition and a second composition, the first composition comprising or consisting of therapeutically effective amount of PD-L1⁺ donor-derived CD4⁺ T-depleted bone marrow cells and the second composition comprising or consisting of the donor organ. In some embodiments, conditioning regimen comprising low-doses of CY, PT, and ATG is administered to the recipient of the transplant composition. In some embodiments, the conditioning regimen is administered to the recipient before the therapeutically effective amount of PD-L1⁺ donor-derived CD4⁺ T-depleted bone marrow cells and before the donor organ. In some embodiments, the conditioning regimen is administered to the recipient after the therapeutically effective amount of PD-L1⁺ donor-derived CD4⁺ T-depleted bone marrow cells and before the donor organ. In some embodiments, the donor organ is a solid organ. In some embodiments, the solid organ is heart, lung, liver, kidney, intestine, pancreas, eye, or skin. In some embodiments, the conditioning regimen is administered to the recipient after the therapeutically effective amount of PD-L1⁺ donor-derived CD4⁺ T-depleted bone marrow cells and after the donor organ. In some embodiments, the treatment composition is used to promote or induce immune tolerance in a recipient.

In some embodiments, the transplant compositions are generated by any of the methods described herein. In some embodiments, the donor comprises any of the characteristics described herein. In some embodiments, the recipient comprises any of the characteristics described herein.

The methods of the present technology are intended for use in a method of promoting or inducing transplant tolerance in a recipient. Transplant tolerance may include, but is not limited to, immunoablation or suppression in respect to immune response against the host-derived tissue while retaining immune competence to non-transplant antigens.

In some embodiments, the present technology provides for a method of promoting or inducing transplant tolerance in a recipient. In some embodiments, transplant tolerance comprises immune tolerance. In some embodiments, immune tolerance is central immune tolerance and peripheral immune tolerance.

The transplant may comprise an organ. In some embodiments, the organ is a solid organ. Nonlimiting examples of solid organs include heart, lung, liver, kidney, intestine, pancreas, and eye. In some embodiments, the organ is skin. In some embodiments, the present technology provides for a method of promoting or inducing transplant tolerance in a recipient. In some embodiments, the method comprises conditioning. In some embodiments, the method comprises HCT. In some embodiments, the method comprises both conditioning and HCT. In some embodiments, the method comprises the COH regimen. In some embodiments, the method further comprises administration of a population of conditioning cells that facilitate engraftment during HCT. In some embodiments, the conditioning cells that facilitate engraftment during HCT is selected from one or more populations of conditioning donor cells selected from donor CD4⁺ T-depleted spleen cells, donor CD8⁺ T cells, and donor Granulocyte colony-stimulating factor (G-CSF)-mobilized peripheral blood mononuclear cells. In some embodiments, the method further comprises measuring PD-L1 expression on a population of CD4⁺ T-depleted donor bone marrow cells and selecting a population of PD-L1⁺ donor-derived CD4⁺ T-depleted bone marrow cells from the population of donor-derived CD4⁺ T-depleted bone marrow cells.

In some embodiments, the transplantation of the population of donor bone marrow cells occurs on the same day as or after the administration of the population of conditioning cells that facilitate engraftment during HCT. In some embodiments, the one or more populations of conditioning donor cells, the donor bone marrow cells, or both are MHC- or HLA-mismatched to the recipient.

The following examples are provided to better illustrate the claimed present technology and are not to be interpreted as limiting the scope of the present technology. To the extent that specific materials are mentioned, it is merely for purposes of illustration and is not intended to limit the present technology. One skilled in the art may develop equivalent means or reactants without the exercise of inventive capacity and without departing from the scope of the present technology.

### EXAMPLES

### Materials and Methods

Mice: Mice were purchased from the National Cancer Institute animal production program (Frederick, MD), The Jackson Laboratory (Bar Harbor, ME), or were bred at the City of Hope Animal Research Center (COH-ARC, Duarte, CA). Detailed information of each strain used is described in Table 1.

TABLE 1
Animals

| Mouse | Cited as |
|---|---|
| BALB/c (H-2d) | BALB/c |
| C57BL/6 (H-2b) | C57BL/6 |
| CD45.1 B6 | CD45.1 |
| MHCII^{-/-} B6 | MHCII^{-/-} |
| Foxp3^{DTR} B6 | DTR |
| PD-L1^{-/-} BALB/c (H-2d) | PD-L1^{-/-} |
| PD1^{-/-} C57BL/6 (B6) | PD1^{-/-} |

Conditioning-induced mixed chimerism: Recipient mice were given daily I.P. injection of cyclophosphamide (CY) at different doses for different models (see details in Table 2), pentostatin (PT) at a dose of 1 mg/kg on D-12, D-9, D-6, D-3 before HCT, and anti-thymocyte globulin (ATG) at a dose of 25 mg/kg on D-12, D-9, D-6 before HCT. On the day of HCT (DO), recipient mice were injected intravenously with bone marrow (BM) and CD4⁺ T-depleted spleen (SPL) cells from donor mice, as shown in Table 2. Twenty-five to thirty days after HCT, peripheral blood was collected from host mice and mixed chimerism status was determined by flow cytometry analysis.

TABLE 2
Induction of MC for different mouse models
using different doses of CY and BM + SPL

| | | BM cells | SPL |
|---|---|---|---|
| Mouse Model | CY (mg/kg × days) | (×10⁶) | (×10⁶) |
| WT BALB/c to WT | (75 × 5) + (50 × 7) | 30 | 40 |
| C57BL/6(B6) | | | |
| WT BALB/c to CD45.1 | (75 × 5) + (50 × 7) | 30 | 40 |
| C57BL/6 (B6) | | | |
| MHCII^{-/-} BALB/c to CD45.1 | (75 × 5) + (50 × 7) | 30 | 40 |
| C57BL/6 | | | |
| PD-L1^{-/-} BALB/c to WT | (75 × 5) + (50 × 7) | 30 | 40 |
| C57BL/6 (B6) | | | |
| WT BALB/c to PD-1^{-/-} | (75 × 5) + (50 × 7) | 30 | 40 |
| C57BL/6 (B6) | | | |
| WT BALB/c to Foxp3^{DTR} | (75 × 5) + (50 × 7) | 30 | 40 |
| C57BL/6 (B6) | | | |
| WT C57BL/6 (B6) to WT | 40 × 12 | 75 | 75 |
| BALB/c | | | |
| MHCII^{-/-} C57BL/6 (B6) to | 40 × 12 | 75 | 75 |
| WT BALB/c | | | |

Skin transplantation (STX): After dorsal skin was harvested from donor mice, connective tissue, fat tissue, and panniculus carnosus were removed from the donor's skin, which was then cut into pieces of 1 cm² grafts. Two pieces of full-thickness skin (1 cm²) grafts were excised from the back of recipient mice, followed by suturing of the allograft onto the graft beds. Skin graft rejection was defined as >90% necrosis of the donor skin tissue after 7-10 days. Hematoxylin & eosin (H&E) staining was performed on paraffin sections of collected skin grafts at the endpoint.

Heart transplantation (HTX): Donor hearts were transplanted heterotopically into recipients by anastomosing the aorta and pulmonary artery of the donor end-to-side to the aorta and inferior vena cava of recipients. Heart transplant rejection was defined as complete cessation of palpable beats, and further verified by laparotomy and H&E staining.

Evaluation and scoring of heart and skin graft histopathology: Hematoxylin & eosin (H&E) staining of skin and heart graft tissues was performed with paraffin sections. The histopathology of tissue slides was evaluated and scored.

Mixed lymphocyte reactions (MLR): For the conditioning alone group or the mixed chimeric group, spleens from recipient mice were collected 30 days or ≥60 days after STX, respectively. As MLR responders, T cells were magnetically purified using CD90.2 MicroBeads, mouse beads T cells were labeled with CFSE, and resuspended in MLR medium (AIM-V™ [Gibco™] supplemented with 10% fetal bovine serum [FBS] and 0.01M HEPES) DCs were enriched from either donor or third-party mice spleen after being digested in digestion buffer (RPMI 1640 containing 0.5 mg/ml collagenase type VIII and 0.5 mg/ml DNase I). DCs were purified using CD11c MicroBeads UltraPure, mouse After purification, the DCs were irradiated and combined with responder T cells in a 96-well round bottom cell culture plate for 5 days. Cells were harvested and analyzed by flow cytometry.

In vivo Treg depletion: Host-specific Treg cells were depleted using an established model. Foxp3⁺ T cells were ablated with diptheria toxin (DT) from Foxp3DTR-KI B6 mice (host). Thirty days after HCT, 20 ug/kg DT was injected to MC mice (i.p.) every 3 days for 30 days except for the first two injections which had a higher dose of 40 ug/kg.

Isolation of lymphocytes from skin: Skin grafts were harvested from recipient mice, minced, and digested with digestion buffer and filtered to generate single-cell suspension. Cell suspensions from each sample were then washed and centrifuged in a Percoll gradient. Lymphocytes were collected from the middle layer and analyzed by flow cytometry.

Dendritic cell (DC) isolation from spleen for DC subset analysis: SPL was harvested and mashed through a 70 μm cell strainer and washed with FACS buffer. CD11C⁺ DCs were isolated using CD11c MicroBeads UltraPure, mouse (Miltenyi Biotec) via magnetic labeling and cell separation. After labeling, DCs were stained with fluorochrome-conjugated antibodies for flow cytometry analysis.

Antibody selection for flow cytometry analysis and cell sorting: Cells were stained with surface marker following incubation with CD16/32 antibody (BioXcell) and aqua viability dye (Invitrogen). Antibodies used are described in Table 3. All intracellular antibody staining including Foxp3 and Helios were performed with the Foxp3/Transcription Factor Staining Buffer Set (eBioscience) following surface marker antibody staining. For intracellular cytokines analysis, cells were stimulated with PMA (Sigma-Aldrich) and lonomycin (Sigma-Aldrich), followed by staining with surface marker antibodies and subsequently with cytokine marker antibodies. Flow cytometry data were acquired using a BD LSRFortessa™ Cell Analyzer. cells of interest were sorted and used for mRNA sequencing.

TABLE 3
Antibody selection for flow cytometry analysis and cell sorting

| Antigen | Conjugation | Manufacturer |
|---|---|---|
| CD45.2 | Brilliant Violet | Biolegend |
| | 605 | |
| H-2Kb | Brilliant Violet | BD |
| | 605 | |
| H-2kd | Brilliant Violet | BD |
| | 605 | |
| TCR-β | Brilliant Violet | Biolegend |
| | 605 | |
| TCR-β | Alexa Fluor 700 | BD |
| CD4 | Alexa Fluor 700 | R&D system |
| CD4 | PE | BD |
| Ly-6G and Ly-6C | PE | BD |
| CD11b | PE | ebioscience |
| CD274 (PD-L1) | PE | ebioscience |
| CD8 | Brilliant Violet | BD |
| | 711 | |
| CD4 | Brilliant Violet | BD |
| | 711 | |
| CD279 (PD-1) | Brilliant Violet | Biolegend |
| | 711 | |
| CD11C | Brilliant Violet | Biolegend |
| | 711 | |
| CD44 | BUV395 | BD |
| CD19 | BUV395 | BD |
| B220 | APC-eFlour ® | ebioscience |
| | 780 | |
| CD62L | APC-eFlour ® | ebioscience |
| | 780 | |
| CD73 | PE-cy7 | ebioscience |
| KLRG1 | PE-cy7 | Biolegend |
| I-Ab | PE-cy7 | Biolegend |
| FR4 | FITC | ebioscience |
| IA/IE | FITC | ebioscience |
| I-Ad | FITC | BD |
| CD304 | PerCP-eFluor ® | ebioscience |
| (Neuropilin-1) | 710 | |
| CD39 | PerCP-eFluor ® | ebioscience |
| | 710 | |
| CD11b | PerCP-Cy5.5 | ebioscience |
| CD8 | PerCP-Cy5.5 | BD |
| Helios | eFluor ® 450 | ebioscience |
| H-2kd | eFluor ® 450 | ebioscience |
| CD45.2 | eFluor ® 450 | ebioscience |
| CD8 | eFluor ® 450 | ebioscience |
| Ly108 | Percific blue | Biolegend |
| FoxP3 | APC | ebioscience |
| CD8 | APC | ebioscience |
| H-2Kb | APC | ebioscience |
| CD45.1 | APC | ebioscience |

Statistics. Statistical analyses were performed using GraphPad Prism version 8.0.1 (San Diego, CA). All quantitative data are shown as mean±SEM. Survival comparisons in different groups were calculated using log-rank test. Comparison of two means was performed using unpaired two-tailed Student's t test, while comparison of multiple means was evaluated using one-way ANOVA. A P value of less than 0.05 was considered as statistically significant.

### Example 1: Conditioning-Induced MCS May Provide Immune Tolerance for Solid Organs

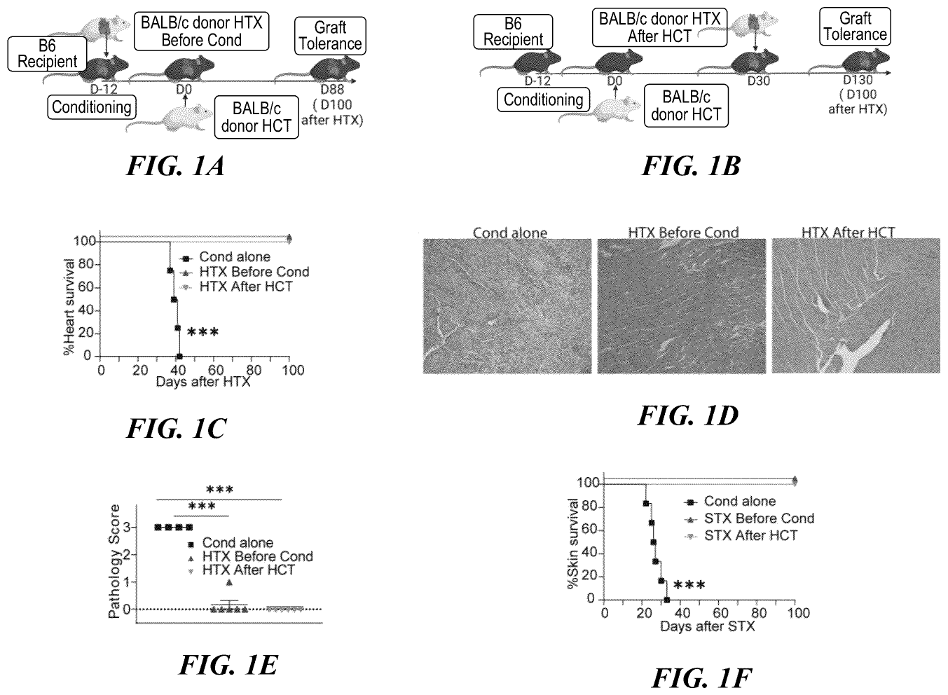

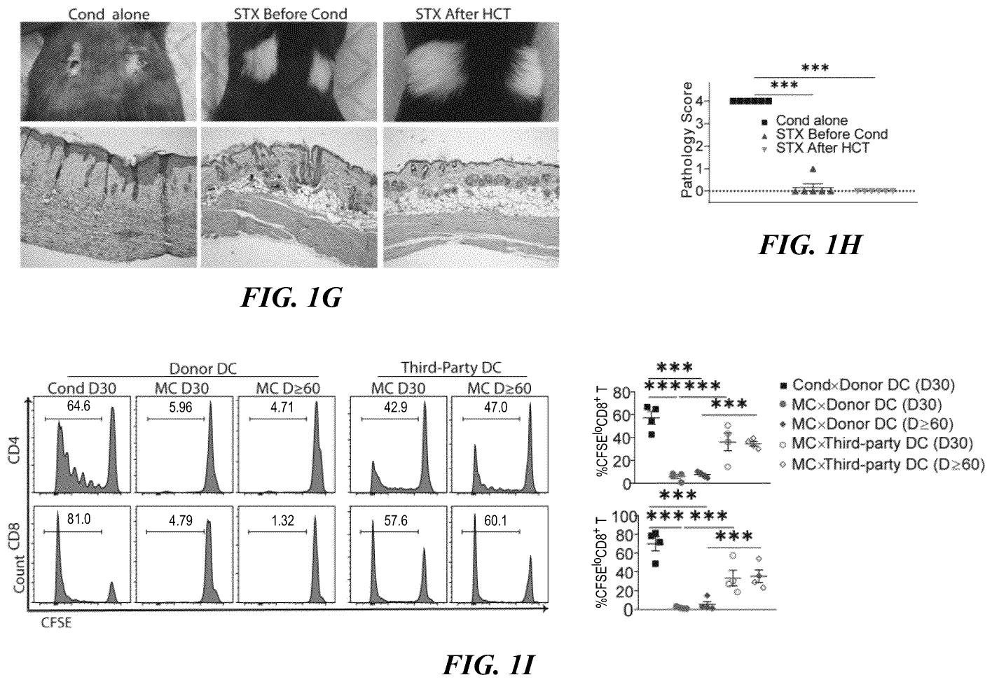

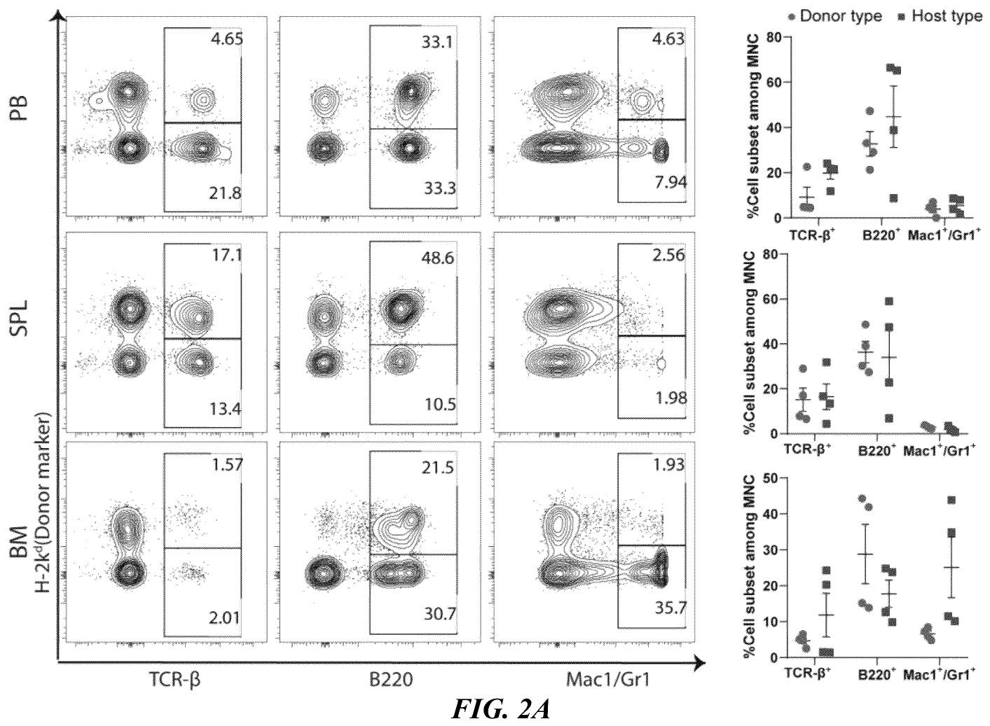

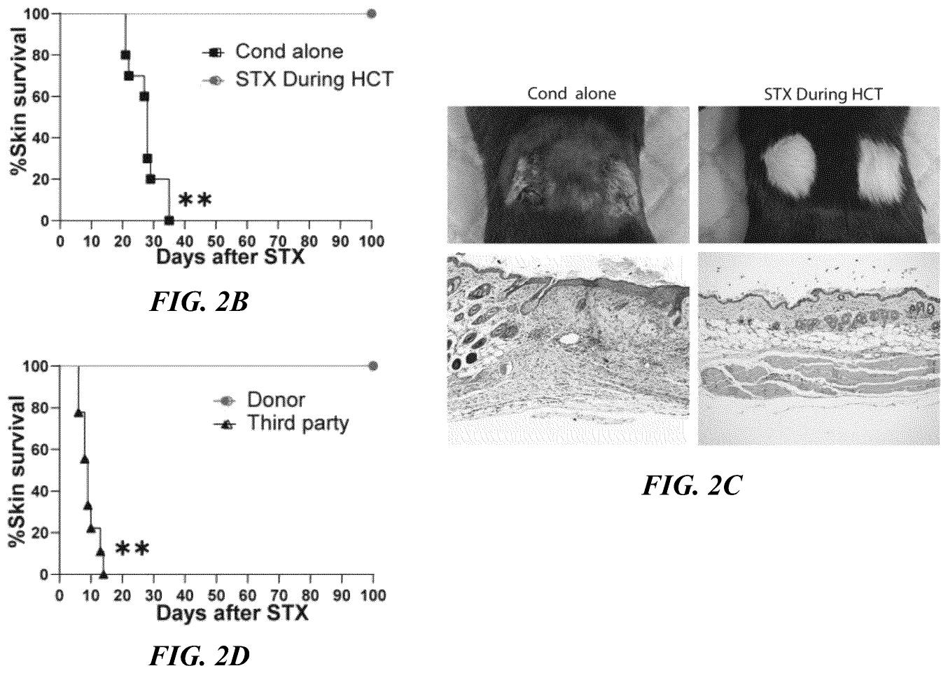

Clinical induction of organ transplant immune tolerance via MC may require performing organ transplantation prior to conditioning and HC. To test whether COH conditioning regimen may induce solid organ transplant immune tolerance, donor-type heart and skin grafts were implanted before, during, or after conditioning and HCT in a murine model of C57BL/6 (B6) recipient and BALB/c donor (FIGS. 1A and 1B). While recipients given Cond Alone rejected donor-type HTX or STX by about day 40 or 30 after conditioning (FIGS. 1C and 1F), MCs with HTX Before Cond, HTX After HCT, STX Before Cond, and STX After Cond all accepted the grafts for more than 100 days without any signs of rejection, as indicated by long-term graft survival and allografts free of lymphocyte infiltration in MC recipients (FIG. 1C-1G). Histopathology was examined and quantified with ISHLT scoring system for heart grafts (1) and Banff scoring system for skin grafts (2). Additionally, histopathology analysis revealed presence of infiltrating lymphocytes or apoptotic parenchymal cells in the HTX or STX of MCs, with pathology scores near 0; in contrast, infiltrating lymphocytes and apoptotic parenchymal cells were abundant in recipient rejecting grafts given Cond Alone, and scores reached the maximal score of 3 for HTX or 4 for STX (FIGS. 1D & E and 1G & 1H). Graft tolerance was again tested when donor grafts were transplanted simultaneously with HCT, where C57BL/6 (B6) recipients were conditioned followed by HCT from BALB/c donors on day 0 to induce MC (FIGS. 2A-2C). MCs also rejected STX from third-party donors (FIG. 2D).

To validate immune tolerance status in MCs in vitro, sorted CD90.2⁺ T cells from recipients given Cond Alone or from MCs that accepted CD90.2⁺ donor-type grafts were stimulated with donor-type DCs in MLR. While the CD4⁺ and CD8⁺ T cells from recipients given Cond Alone proliferated vigorously, those derived from MCs showed low proliferation in response to donor-type DC stimulation but proliferated vigorously in response to third-party DC stimulation as shown by measurements at both day 30 and day 60 (FIG. 1I). These results indicate that conditioning-induced MCs may provide immune tolerance to grafts transplanted before, during, and after conditioning and HCT.

### Example 2: Donor Hematopoietic Cell Expression of MHCII May not be Required for Conditioning-Induced MCs

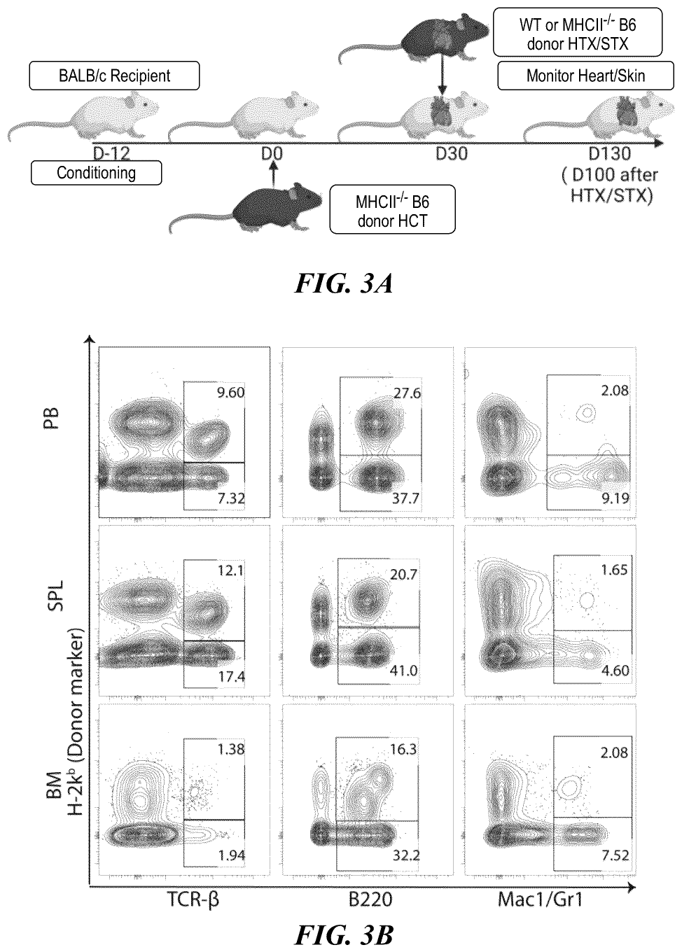

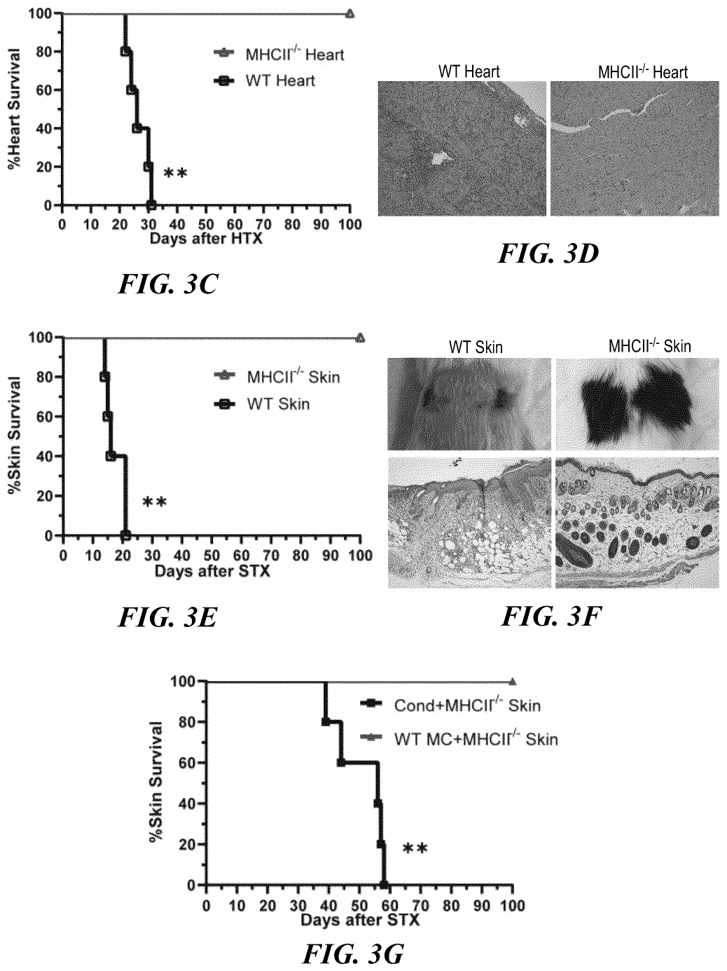

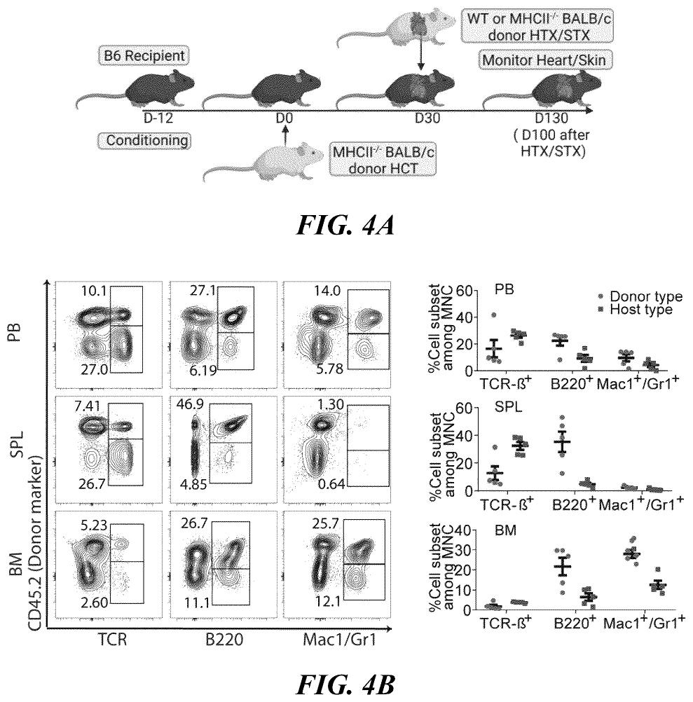

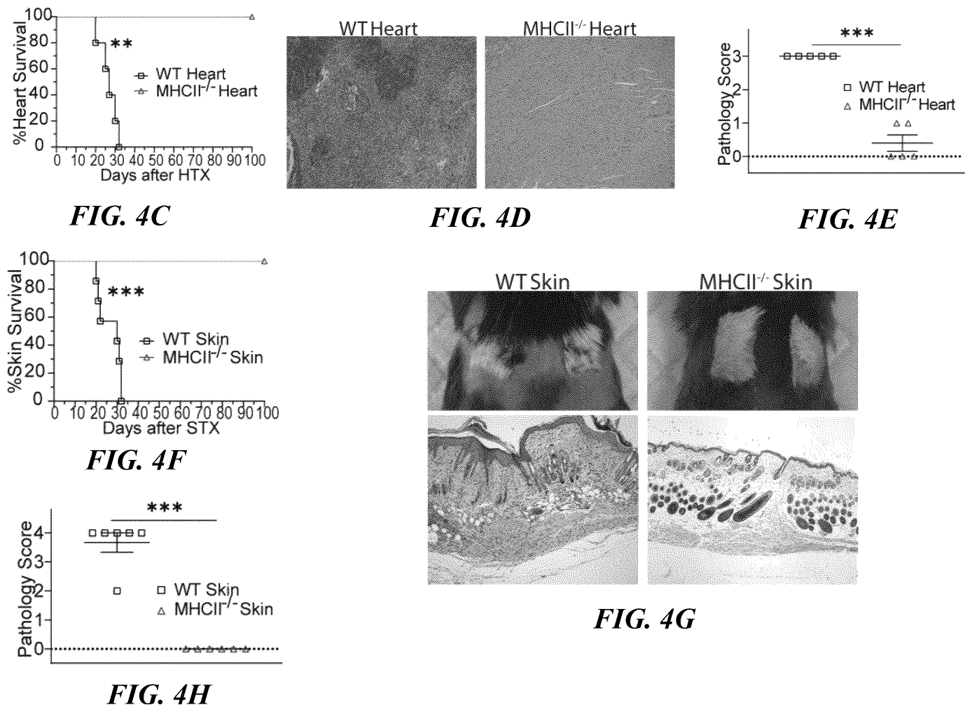

Donor-type dendritic cell (DC) expression of MHCII may play a critical role in the central tolerance of conditioning-induced MCs. Additionally, MHCII^{-/-} donor HCT was not able to induce stable MC under a conditioning regimen with co-stimulatory blockade, as described in Example 1. To test whether conditioning-induced MCs permits induction of stable MC and donor-type organ transplant tolerance with MHCII^{-/-} donors, C57BL/6 or BALB/c recipients were conditioned with COH conditioning regimen and infused with CD4⁺ T-depleted splenic cells and whole bone marrow cells from MHCII^{-/-} BALB/c or C57BL/6 donors (FIGS. 3A and 4A). CD45.1 B6 recipients were conditioned and followed by HCT from MHCII^{-/-} or WT BALB/c donors on day 0 to induce MHCII^{-/-} or WT MC and then transplanted with either STX or HTX from MHCII^{-/-} or WT donors on day 30 after HCT. Stable MC were observed in both C57BL/6 and BALB/c recipients given MHCII^{-/-} donor SPL and BM cells (FIGS. 3B and 4B). Moreover, MHCII^{-/-} MCs accepted donor-type MHCII^{-/-} HTX and STX without any signs of rejection, while rejecting WT MHC^{+/+} donor-type graft, as indicated by survival curves and histopathology score (FIGS. 3C-3F and 4C-4H). Additionally, WT MCs also accepted MHCII^{-/-} grafts without any signs of rejection (FIG. 3G). These results indicate that MHCII expressed by HCT is not required for conditioning-induced MCs that provide immune tolerance to donor-type MHCII^{-/-} organ transplants.

### Example 3: Residual Donor-Reactive T Cells in the Periphery of Conditioning-Induced MCs May Become Anergic in a Donor-Type MHCII-Dependent Manner

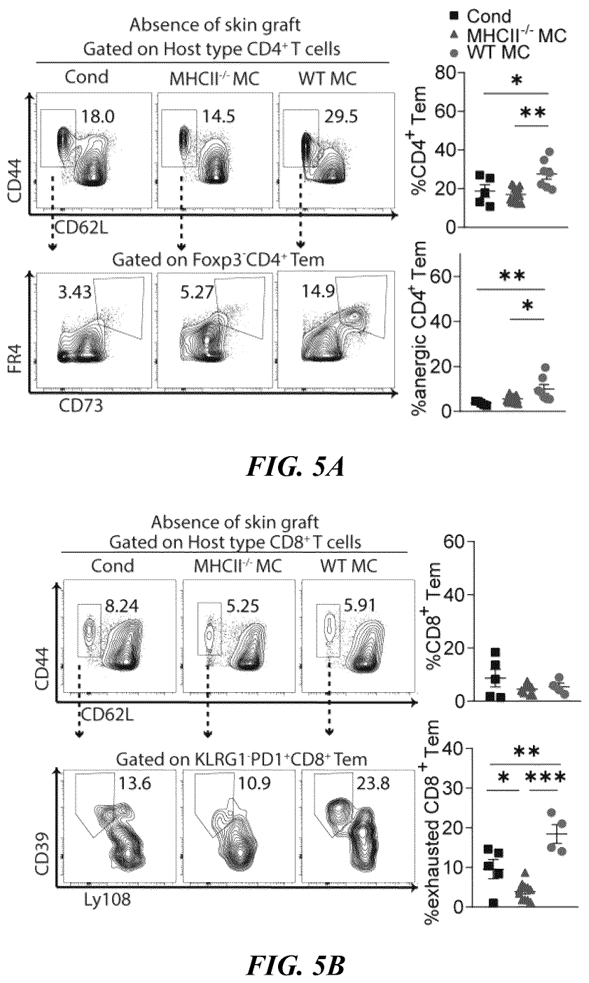

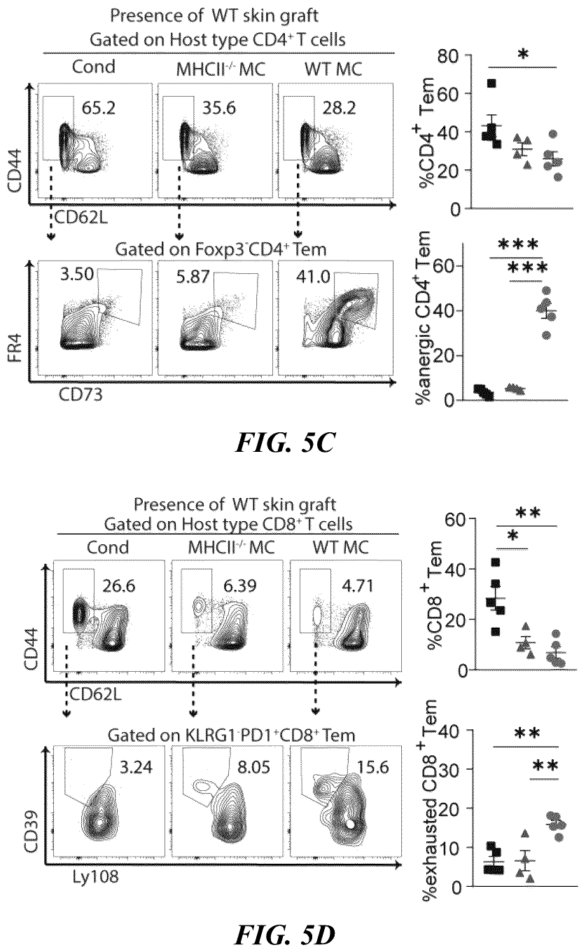

Donor reactive T cells may be deleted in the MC thymus in a donor-MHCII dependent manner. It is unknown whether anergic/exhausted residual donor-reactive T cells are in MCs tolerant to donor-type organ transplant. Therefore, host-type T cell activation and was compared with anergy/exhaustion status in SPL of WT MCs, MHCII^{-/-} MCs, and mice given Cond Alone. In comparison to mice given Cond Alone or MHCII^{-/-} MCs, WT MCs displayed significant increase in percentage of CD62L⁻CD44⁺CD4⁺ effector/memory Tem cells in the SPL and a significant increase in CD73^{hi}FR4^{hi}CD4⁺ anergic subset among the Foxp3⁻CD4⁺ Tem cells on day 30 after HCT (FIG. Cond Alone, WT MC, or MHCII^{-/-} MC without or with STX were analyzed with flow cytometry. Although the WT MC did not show significant increase in the percentage of CD8⁺ Tem cells, there was a significant increase in percentage of KLRG1⁻PD-1⁺ Ly108⁻CD39⁺ exhausted subset among the CD8⁺ Tem cells, as compared to Cond Alone or MHCII^{-/-} MCs (FIG. 5B). These results suggest that residual donor reactive CD4⁺ and CD8⁺ host-type T cells exist in the periphery of WT MCs and are in anergic status.

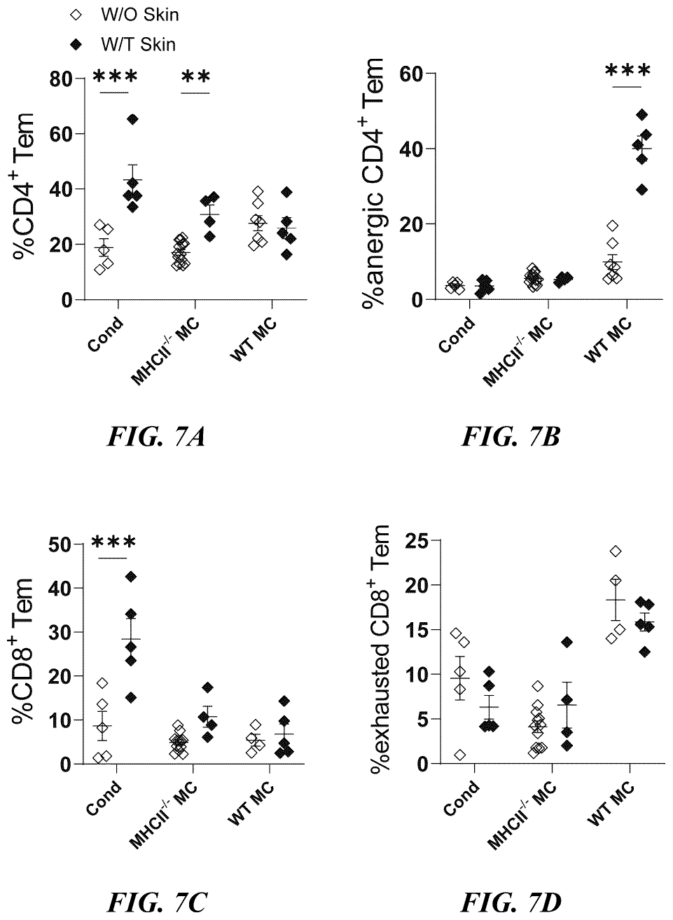

Mice were also challenged with WT donor-type STX. Percentage of CD4⁺ Tem cells in tSPL of mice given Cond Alone and in the spleen of MHCII^{-/-} MCs was increased by WT donor-type STX, but not WT MCs (FIGS. 5C and 7A). In contrast, the percentage of anergic CD73^{hi}FR4^{hi} subset among Foxp3⁻CD4⁺ Tem cells in WT MCs increased as compared with those of mice given Cond Alone or MHCII-MCs (FIG. 5C) as well as compared with WT MC in the absence of STX challenge (FIG. 5A). Percentage of anergic CD4⁺ Tem cells in MCs with STX was greater than in MCs lacking STX (FIG. 7B). CD8⁺ Tem cells increased in the Cond when challenged with donor-type STX, although not in WT or MHCII^{-/-} MCs (FIG. 7C). Donor-type STX challenge increased percentage of exhausted KLRG1⁻PD-1⁺ Ly108⁻CD39⁺ subset (3) among CD8⁺ Tem cells was significantly increased in WT MCs, as compared to MHCII^{-/-} MCs or mice given Cond Alone (FIGS. 5D and 7D). Anergic CD73^{hi}FR4^{hi}CD4⁺ T and exhausted KLRG1⁻PD⁻1⁺Ly108⁻CD39⁺CD8⁺ T cells may reduce cytokine production (i.e., IL-2 and IFN-gamma) and reduced proliferation (3, 4), indicating potential donor reactive CD4⁺ and CD8⁺ T cells with phenotype of anergy/exhaustion in WT MCs periphery. When challenged by donor-type solid organ transplant, residual CD4⁺ T cells may transiently expand and become anergic CD73^{hi}FR4^{hi} phenotype again in a donor-type MHCII-dependent manner. This indicates that there may be residual donor reactive T cells in the periphery of WT MCs which become anergic/exhausted in a donor-type MHCII expressing APC-dependent manner when challenged by donor-type solid organ transplant.

### Example 4: Residual Donor MHCII-Reactive CD4+ Host-Type T Cells in the Periphery of Conditioning-Induced MCs May Differentiate into pTreg Cells in Response to Donor-Type STX Stimulation

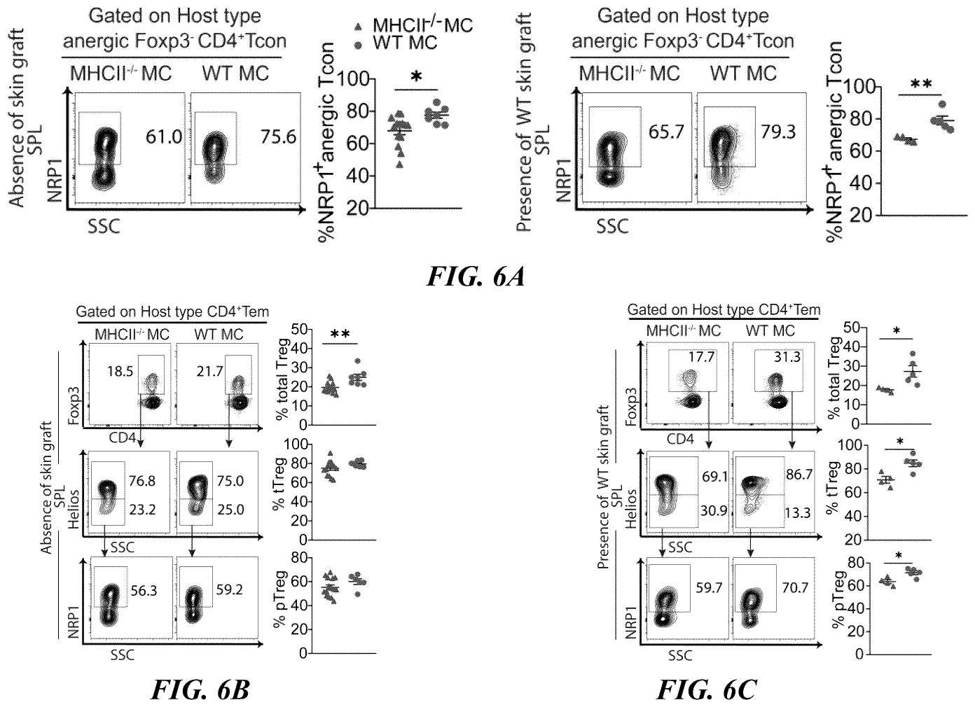

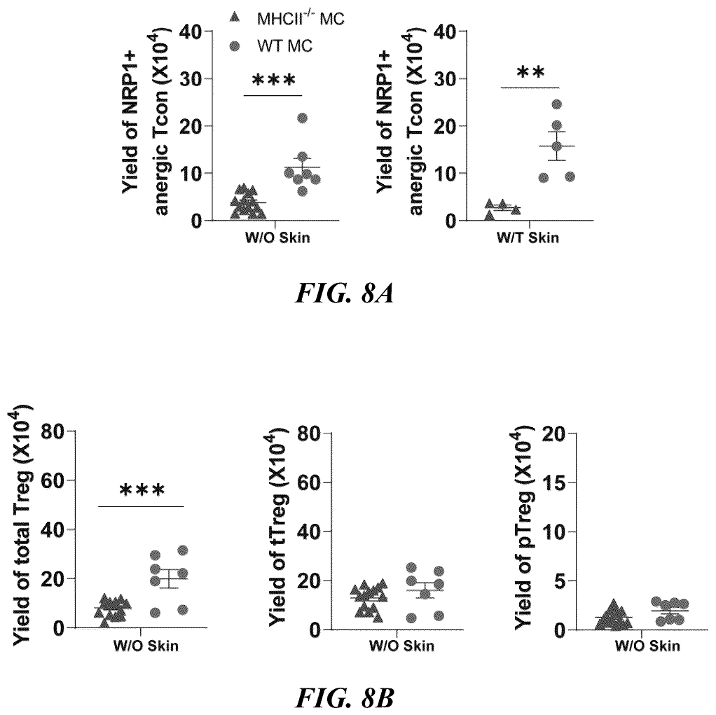

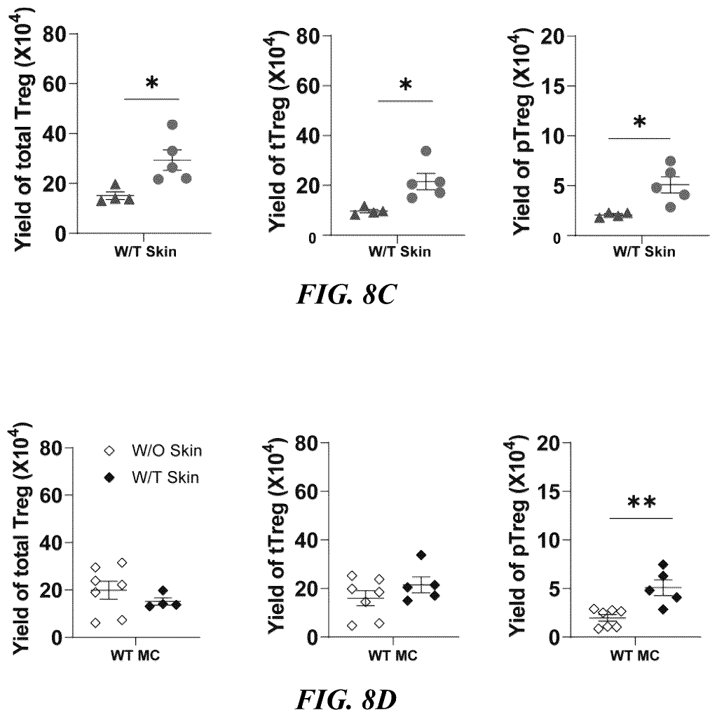

Neurophilin 1 (NRP1) may be involved in regulatory T (Treg) cell development, and lack of NRP1 on CD4⁺ Tcon cells may prevent Treg but augments Th17 differentiation and anergic NRP1⁺CD73^{hi}FR4^{hi}CD4⁺ T cells may be the precursor of NRP1⁺ pTreg cells. After donor-type STX, D73^{hi}FR4^{hi}CD4⁺ T cell expanded in the QT MCs (FIG. 5C). Additionally, NRP1⁺CD73^{hi}FR4^{hi}CD4⁺ pTreg precursors and NRP1⁺ pTreg cells were expanded in SPL of the WT MCs with or without donor STX (FIGS. 6A and 8A). 30 days after HCT and STX, CD45.1+ host-type CD4+ T subsets from the spleen or draining lymph nodes (LN of WT MC or MHCII^{-/-} MC without or with STX were analyzed with flow cytometry, TCR-CDR3-Seq, and single cell RNA-Seq.

CD73^{hi}FR4^{hi}CD4⁺ T cell expansion occurred in the WT MCs after donor-type STX in comparison to that of MHCII^{-/-} MCs (FIG. 5C). Percentage of total Foxp3+ Treg cells (% total Treg) among CD4+ Tem cells, percentage of Helios+ tTreg cells (% tTreg) among total Treg cells, and percentage of Helios−NRP1+pTreg cells (% pTreg) among Helios-Treg cells, and total Treg, tTreg and pTreg cells were compared. SPL of MCs without STX, displayed increased percentage and Treg cells yield relative to MHCII^{-/-} MCs, there was no significant difference in pTreg cells percentage or yield (FIGS. 6B and 8B). WT MCs SPL with donor-type STX demonstrated increased percentage and yield of total Treg, tTreg, and pTreg cells relative to MHCII^{-/-} MCs (FIGS. 6C and 8C). MCs with STX increased SPL yield of pTreg cells but not tTreg cells (FIG. 8D), indicating MHC-mismatched MCs donor reactive CD4⁺ T cells may differentiate into pTreg cells in a donor-type MHCII-dependent manner when transplanted with a donor-type organ graft.

### Example 5: Depletion of Host-Type Treg Cells May Result in Decrease of Donor-Type CD8+ DCs, Down-Regulation of DC PD-L1, and Rejection of Donor-Type Skin Graft

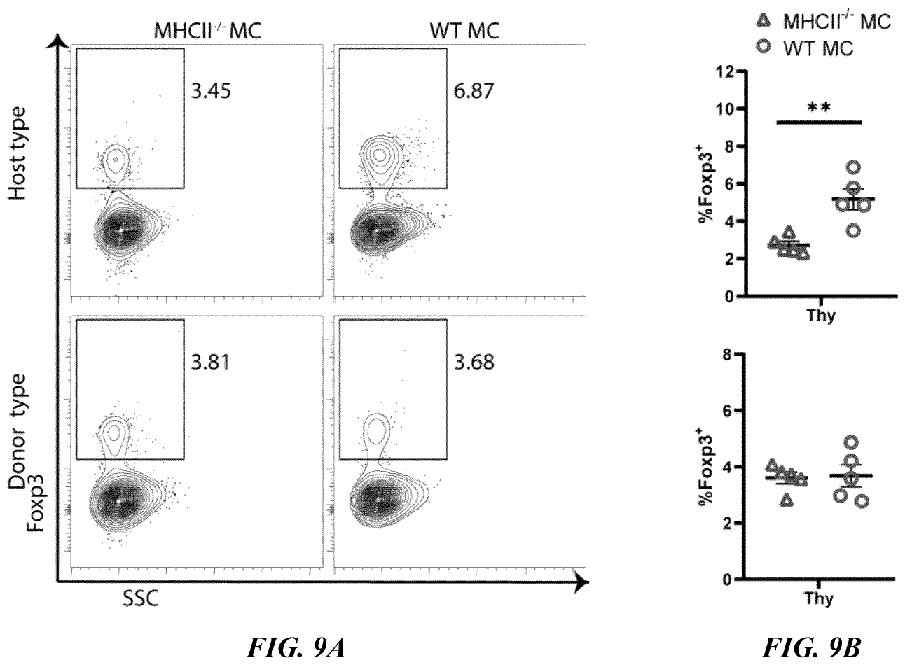

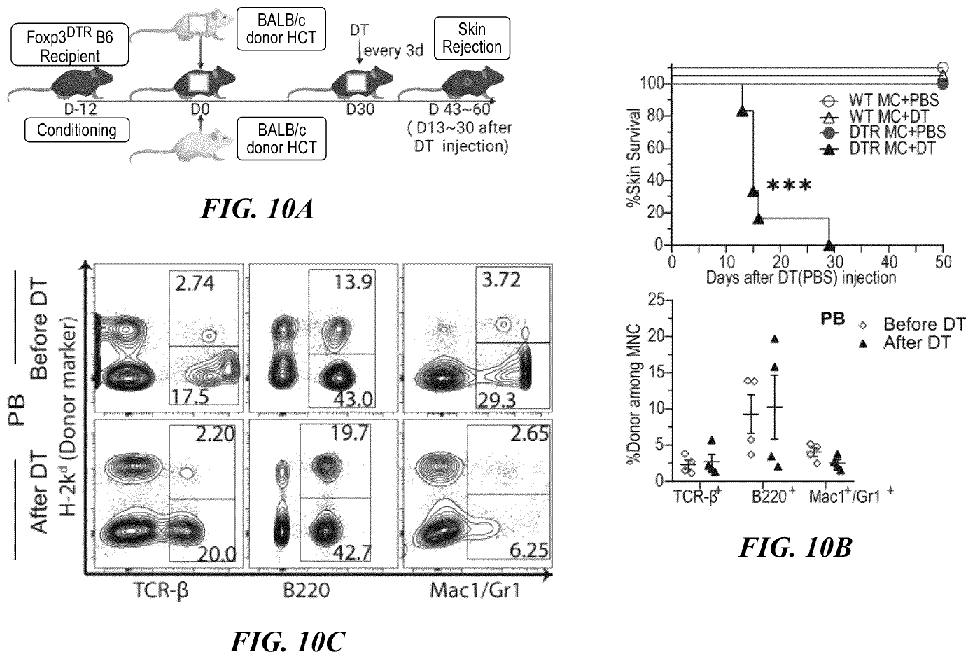

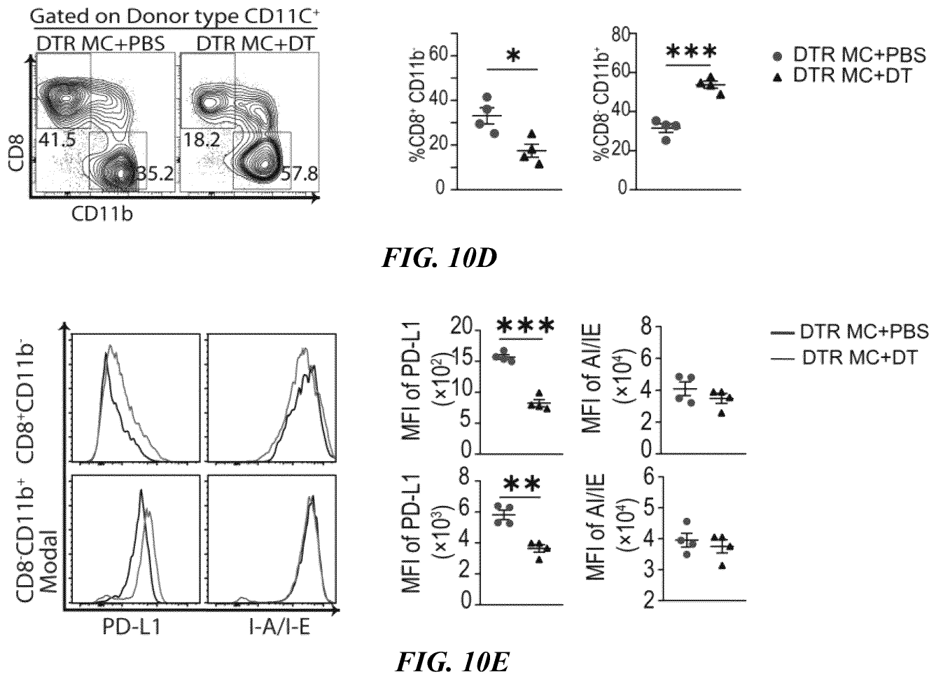

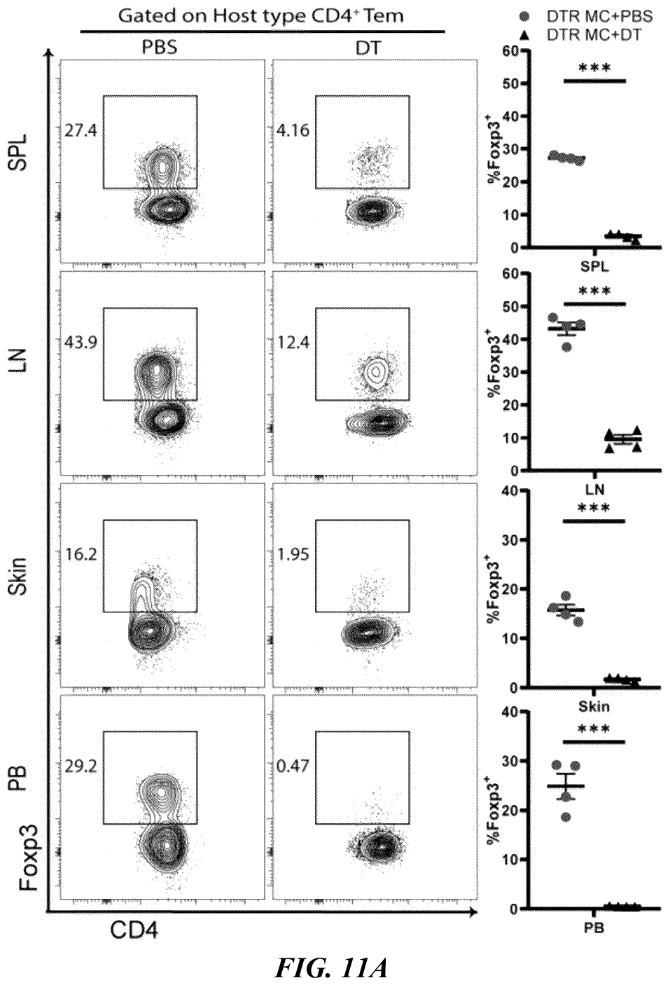

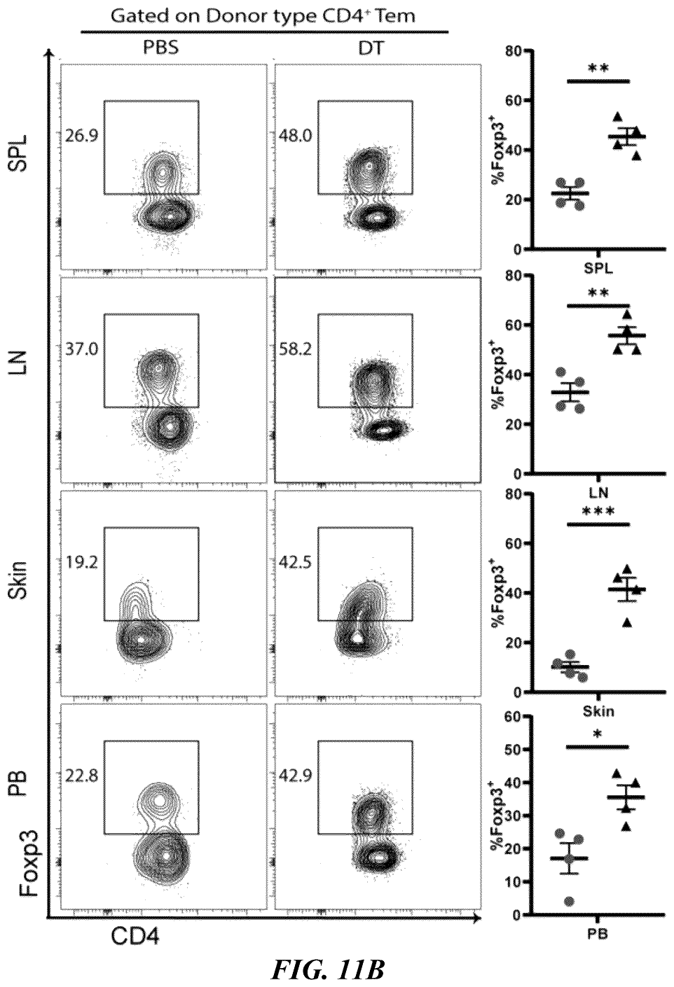

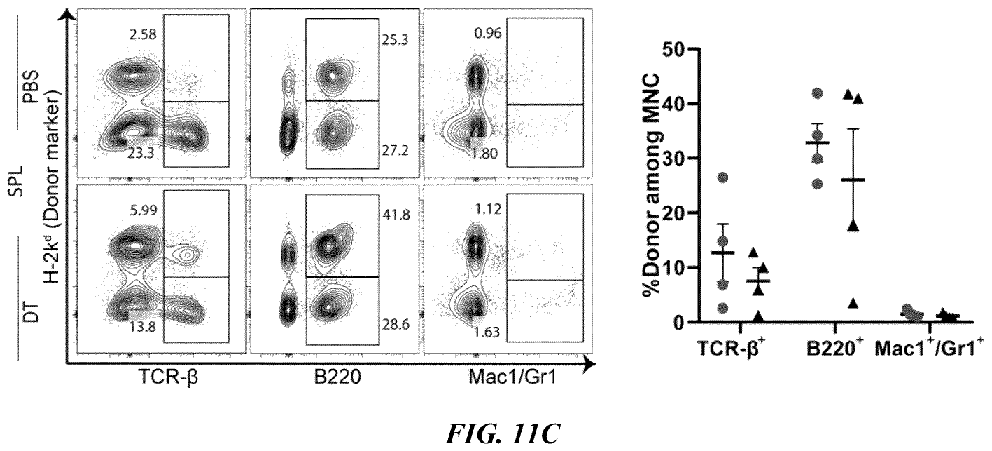

pTreg differentiation may require T cell interaction with tolerogenic DCs such as CD8⁺CD11b⁻ DCs that express high levels of PD-L1. Moreover, tTreg cells may play an important role in maintaining tolerogenic DCs. Enhanced thymic generation of host-type but not donor-type tTreg cells was observed in WT MCs, as compared to MHCII^{-/-} MCs (FIGS. 9A and 9B). It was assessed whether host-type Treg cells were required for maintaining stable mixed chimerism, donor-type CD8⁺ DCs and their expression of PD-L1, and donor-type organ transplant tolerance. Accordingly, Foxp3DTR knock-in mice were conditioned and induced for WT MC via HCT and implanted with STX from donor-type mice on day 0 after HCT. 30 days after induction of MC and STX, recipients were injected with diphtheria toxin (DT) or PBS every 3 days for 15-30 days, starting on day 30 after HCT (FIG. 10A). WT B6 mice with conditioning-induced MCs given STX and treated with PBS were labeled WT MC+PBS; WT B6 mice with conditioning-induced MCs and given STX and treated with DT are labeled WT MC+DT; Foxp3DTR mice with conditioning-induced MCs and given STX and treated with PBS were labeled DTR MC+PBS; Foxp3DTR mice with conditioning-induced MCs and given STX and treated with DT were labeled DTR MC+DT. WT MC+DT mice were used as additional controls. Injection of DT resulted in rejection of STX in all Foxp3DTR MCs (6/6), and no rejections were observed in DTR MC+PBS nor WT MC+DT mice (FIG. 10B). Injection of DT reduced host-type Treg cells and increased donor-type Treg cells in the PB, SPL, LN, and STX (FIGS. 11A and 11B). Injection of DT did not have significant impact on the MC status measured before and after DT treatment in the PB as well as in SPL (FIGS. 10C & FIGS. 11C). Host-type Treg cell depletion reduced percentage of donor-type CD8⁺CD11b DC subsets; and residual donor-type CD8⁺ DCs reduced expression levels of PD-L1, as measured by MFI (FIGS. 10D and 10E). Host-type Treg cell depletion increased percentage of CD8⁻CD11b+DC subset, and the CD11b⁺ DCs also reduced PD-L1 expression levels, as measured by MFI (FIGS. 10D and 10E). Moreover, depletion of host-type Treg cells altered MHCII I-A/I-E expression levels of donor-type CD8⁺ or CD11b⁺ DC subsets (FIG. 10E), indicating in the conditioning-induced MCs, host-type Treg cells may be required for maintaining expansion of donor-type tolerogenic CD8⁺ DCs and expression of PD-L1 as well as donor transplant tolerance, although not required for maintaining stable MC.

### Example 6: Donor Hematopoietic Cell PD-L1 and Host-Type T Cell PD-1 May be Required for Expansion of Host-Type pTreg Cells and Donor-Type Transplant Tolerance

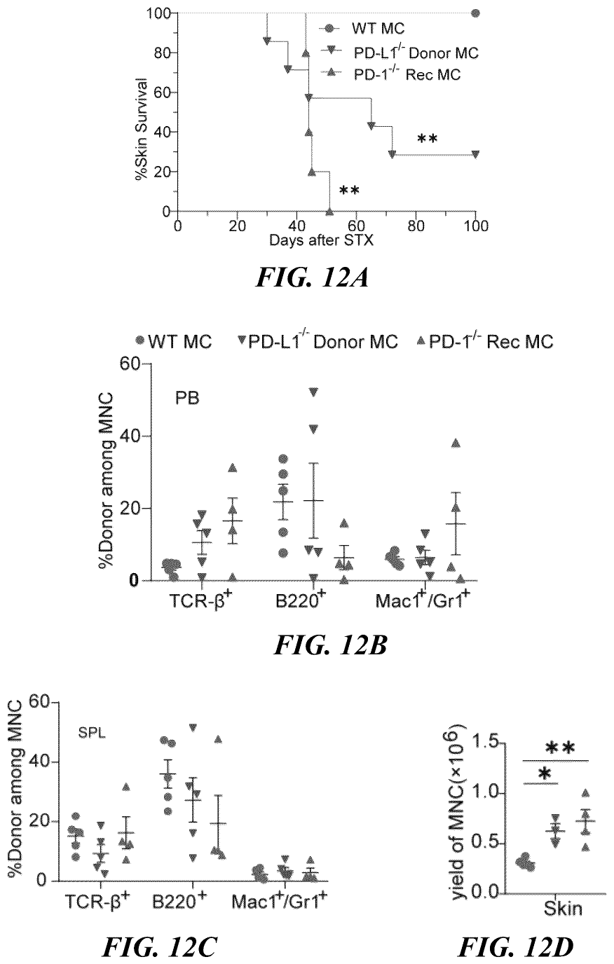

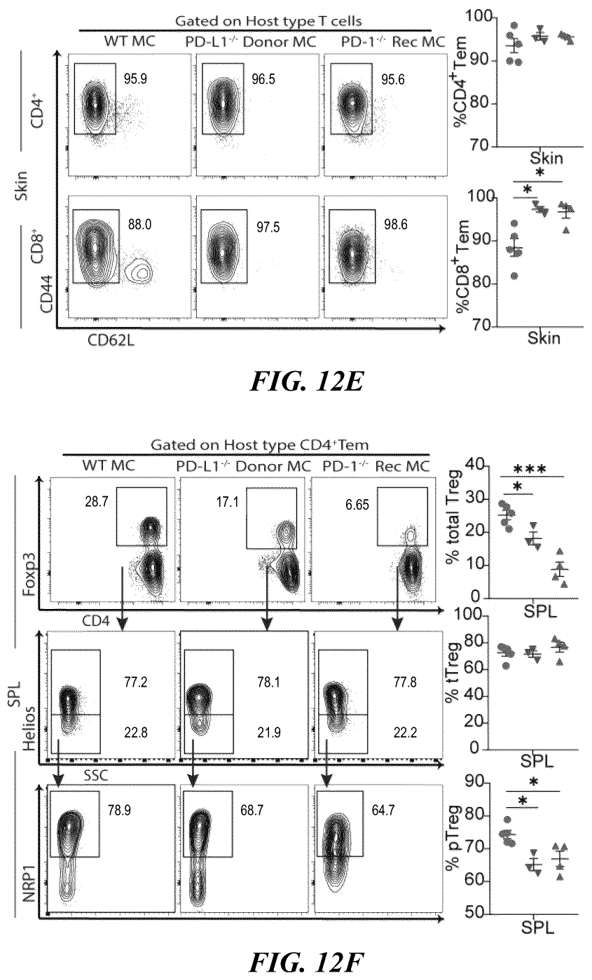

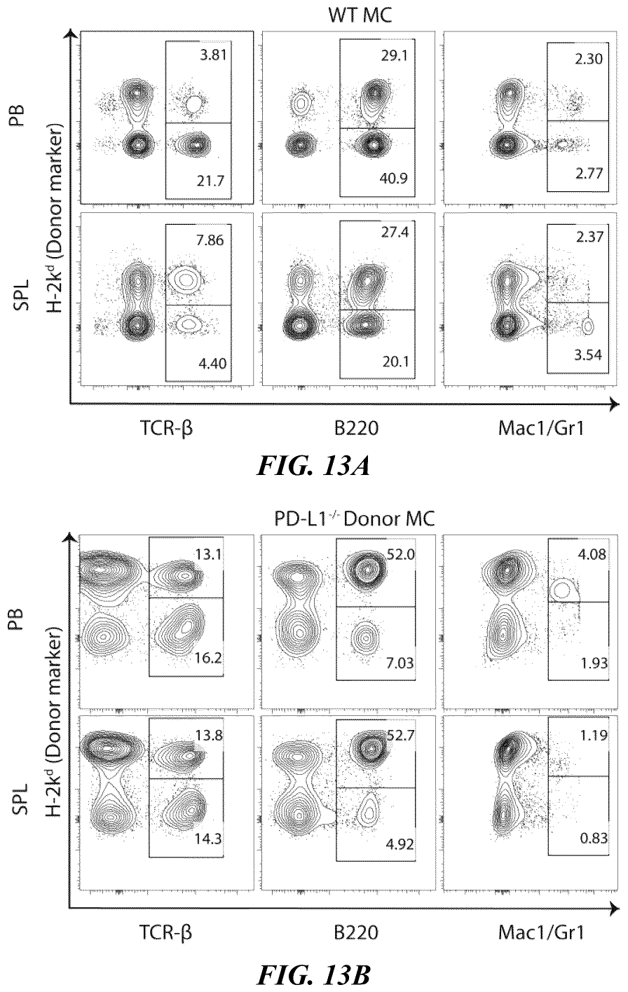

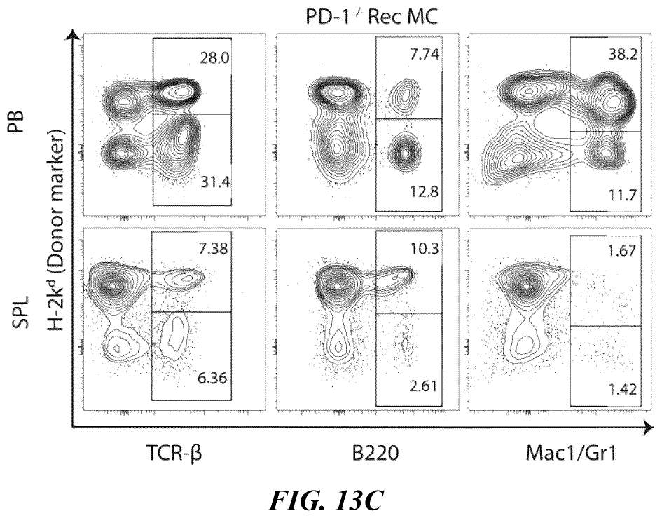

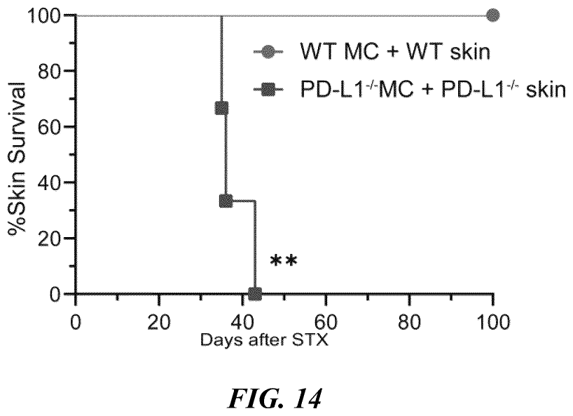

Although PD-L1/PD-1 interaction plays an important role in induction of host-reactive donor-type T cell anergy/exhaustion and generation of pTreg cells in murine GVHD models, the impact of PD-L1/PD-1 interaction on conditioning induction of stable MC, generation of donor-reactive host-type pTreg cells, and donor-type organ transplant tolerance remains unclear. Therefore, it was assessed whether conditioning-induced MCs may be induced with PD-L1/donor hematopoietic cells or in PD-1/recipients. WT or PD-1^{-/-} C57BL/6 mice were conditioned and transplanted with SPL and BM cells from PD-L1^{-/-}or WT donors to induce MC. 30 days after HCT, WT recipients given either WT donor hematopoietic cells (WT MC) or PD-L1/donor hematopoietic cells (PD-L1^{-/-} Donor MC), or PD-1/recipients given WT donor hematopoietic cells (PD-1^{-/-}Rec MC) were implanted with STX from WT donor-type BALB/c mice. All MCs were subjected to STX from WT BALB/c donors on day 30 after HCT. While all (6/6) WT MCs accepted STX without any signs of rejection for more than 100 days, most (5/7) of PD-L1^{-/-}donor MCs rejected STX between about 30-70 days after transplantation (FIG. 12A). All (5/5) of PD-1^{-/-}Rec MC rejected STX between about 40-50 days after transplantation (FIG. 12A). No significant difference in donor chimerism levels among the three groups were observed in PB or SPL cells at the end of observation (FIGS. 12B, 12C, and 13A-13C). Additionally, PD-L1/donor MC also rejected PD-L1^{-/-}donor-type skin graft, where WT B6 recipients underwent conditioning followed by HCT from either WT (WT MC) or PD-L1^{-/-}BALB/c (PD-L1^{-/-}MC) donors and then subjected to WT (WT skin) or PD-L1^{-/-}BALB/c (PD-L1^{-/-}skin) STX during HCT on day 0, (FIG. 14), indicating that donor-type APC PD-L1 interaction with host-type T cell PD-1 may be required for organ transplant tolerance in conditioning-induced MCs, although not required for induction of stable MC.

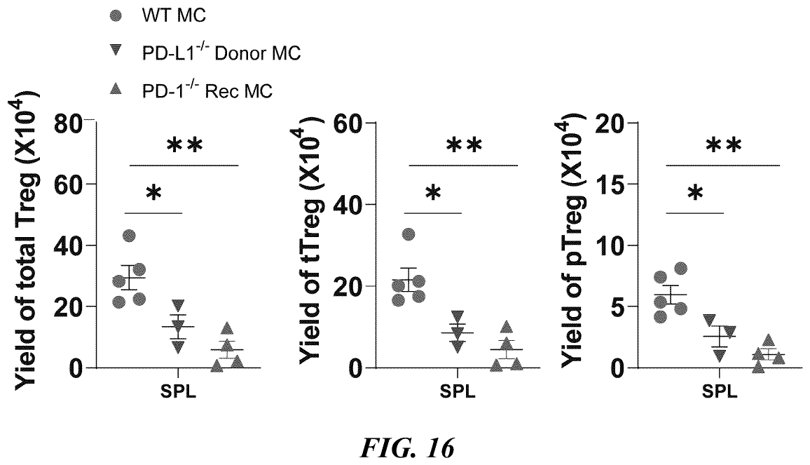

To explore mechanisms of STX rejection in PD-L1/donor MCs or PD-1^{-/-}Rec MCs, lymphocyte infiltration severity was assessed by measuring total mononuclear cells (MNC) and CD4⁺ and CD8⁺ Tem cell percentage. Compared to tolerant WT MCs, MNC yield increased in the rejected STX from PD-L1^{-/-}donor MCs or PD-1^{-/-} Rec MCs (FIG. 12D); and increase in percentage of CD8⁺ Tem, although not CD4⁺ Tem cells, among host-type T cells (FIG. 12E). Percentages of host-type total Treg, tTreg and pTreg cells among CD4⁺ Tem cells in SPL were analyzed. As compared to the tolerant WT MCs, there was a significant reduction in percentage of total Treg cells among host-type CD4⁺ Tem cells, with reduced percentage of pTreg cells but not tTreg cells in the spleen of PD-L1^{-/-}donor MCs and PD-1^{-/-}Rec MCs (FIG. 12F). Yield of Foxp3+CD4+ total Treg cells, Helios+ tTreg cells, and Helios-NRP1+pTreg cells in SPL for each group were calculated. Total Treg, tTreg, and pTreg yields were reduced in PD-L1/donor MCs and PD-1^{-/-}Rec MCs (FIG. 16), indicating STX rejection in the absence of donor APC PD-L1 interaction with host PD-1 in the conditioning-induced MCs is associated with host-type pTreg cell reduction.

### Example 7: Donor-Type Graft Expression of PD-L1 May be Required for Organ Transplant Tolerance and Expansion of Host-Type pTreg Cells in Conditioning-Induced MCs

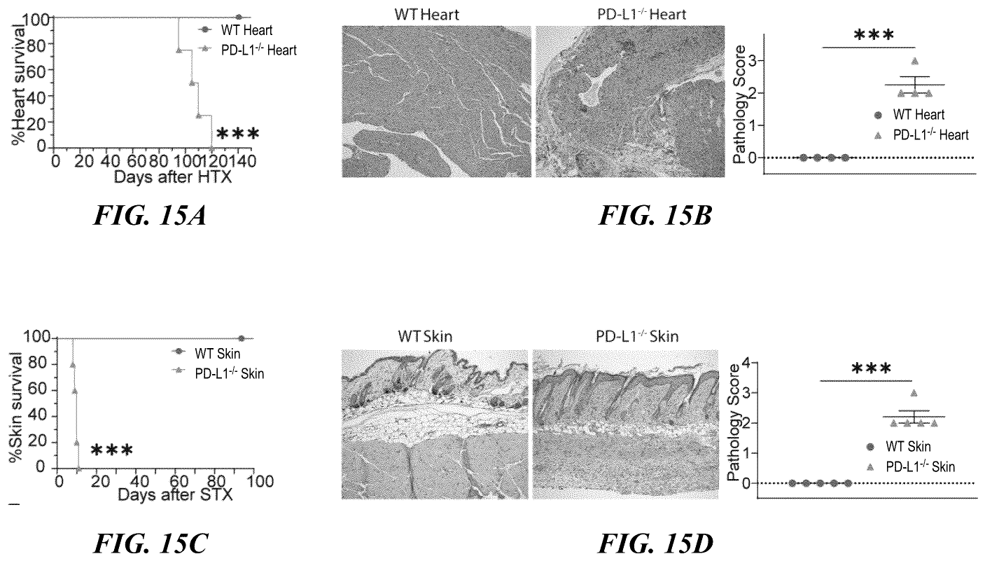

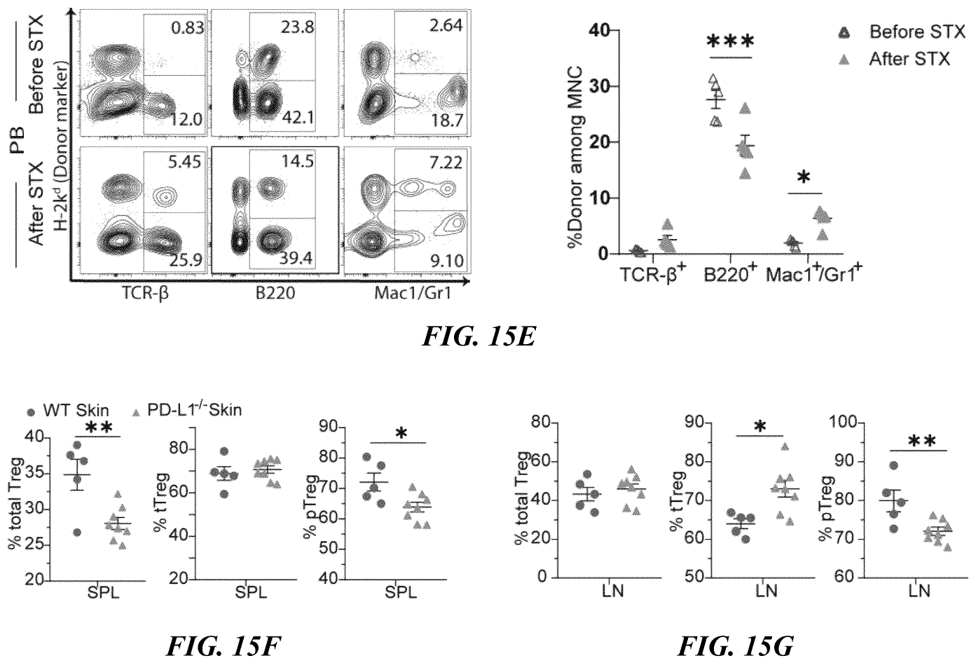

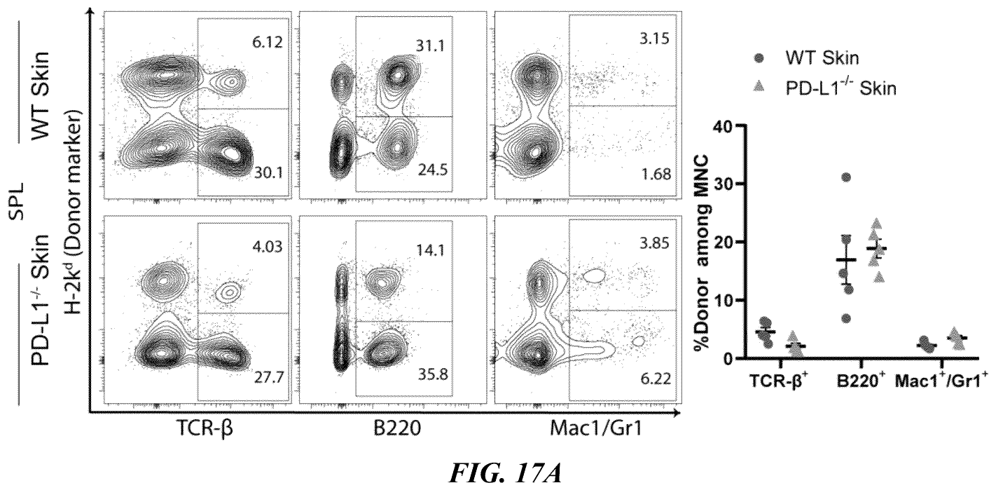

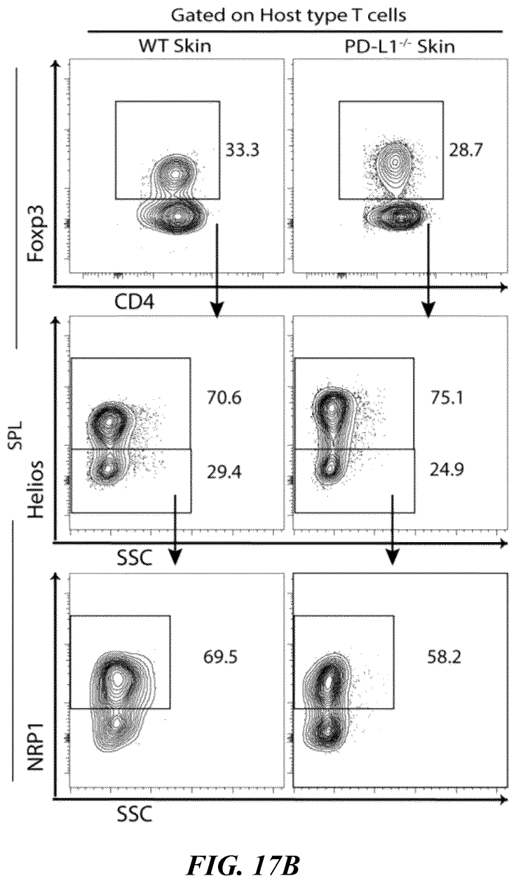

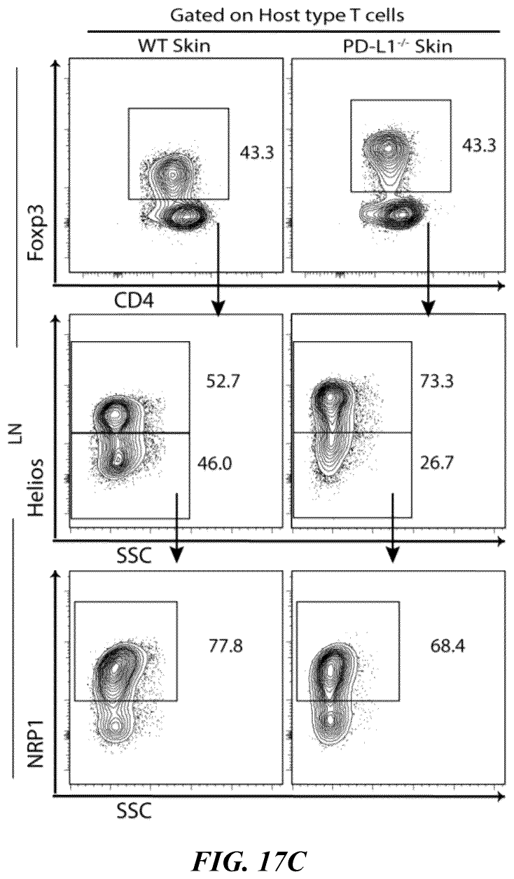

Because donor-type hematopoietic cell expression of PD-L1 was required for organ transplant tolerance in conditioning-induced MCs (FIGS. 12A-12F), it was tested whether donor-type organ expression of PD-L1 was also required for transplant tolerance in the conditioning-induced MCs. Accordingly, WT MCs on day 60 after HCT were transplanted with WT or PD-L1^{-/-} donor-type heart or skin grafts. All (4/4) WT HTX survived for more than 150 days without signs of rejection as indicated by strong graft heartbeat and free of tissue lymphocyte infiltration (FIGS. 15A and 15B). All (4/4) PD-L1/grafts were rejected between about 100 to about 120 days after HCT, as indicated by tissue lymphocyte infiltration, and by the loss of graft heartbeat and pathology score of 2-3 and moderate parenchymal apoptosis (FIGS. 15A and 15B). While all (5/5) WT donor-type STX survived for more than 100 days without signs of rejection or tissue infiltration, all (5/5) PD-L1/STX was rejected by about 10 days after skin transplantation with strong infiltration being observed and epidermal hyperplasia and histopathology score 2-3 (FIGS. 15C and 15D). PD-L1^{-/-}donor-type STX rejection (transplanted on day 60 after HCT) did not reduce the percentage of donor-type T or myeloid cells, although it reduced in percentage of donor-type B cells in the MCs, as indicated by MC status among blood cells before and after transplantation (FIG. 15E). However, no difference in MC was observed among SPL in recipients transplanted with WT or PD-L1^{-/-} graft (FIGS. 17A-17C).

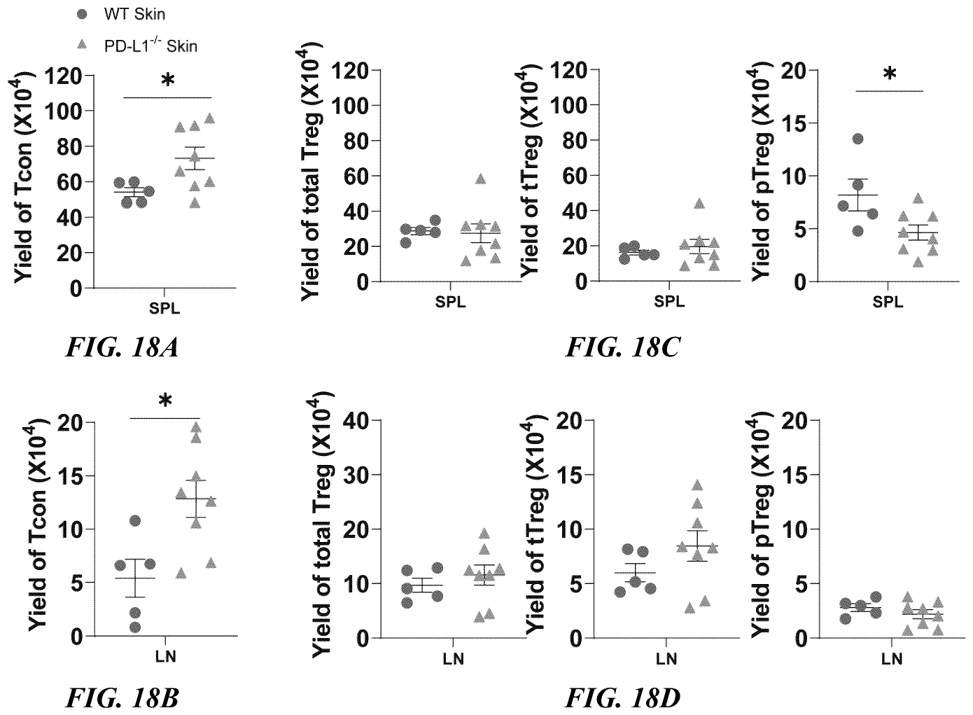

As compared to tolerant MCs with WT STX, MCs showed increase in host-type CD4⁺ Tcon cells in SPL and LN (FIGS. 18A and 18B), and MCs rejecting PD-L1/STX reduced percentage of total Treg cells among splenic CD4⁺ Tem cells with reduced percentage of pTreg cells but no significant difference in percentage of tTreg cells (FIGS. 15F and 18C). MCs with PD-L1/STX also showed increased of host-type CD4⁺ Tcon cells in SPL and LN, especially in the draining LN (FIGS. 18A and 18B). In the draining LN, however, there was no difference in percentage of total Treg cells among CD4⁺ Tem cells, although there was an increase in percentage of tTreg cells and reduction in percentage of pTreg cells (FIGS. 15E, 15G, 18C, and 18D).

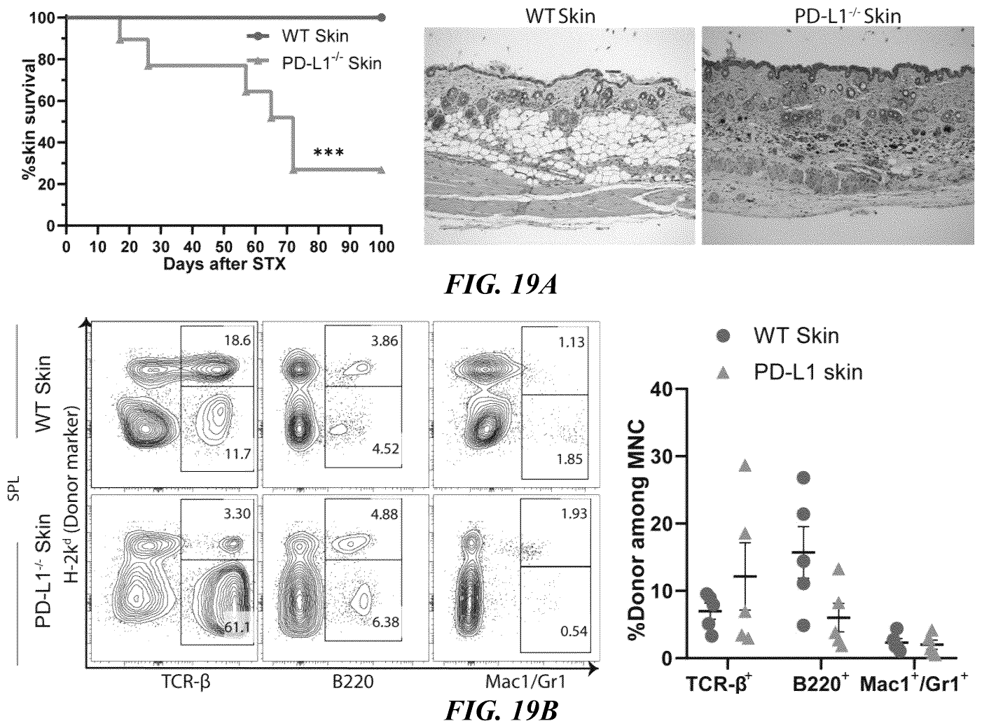

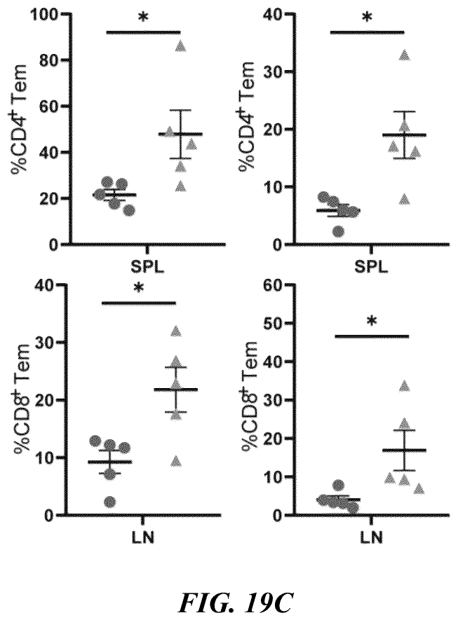

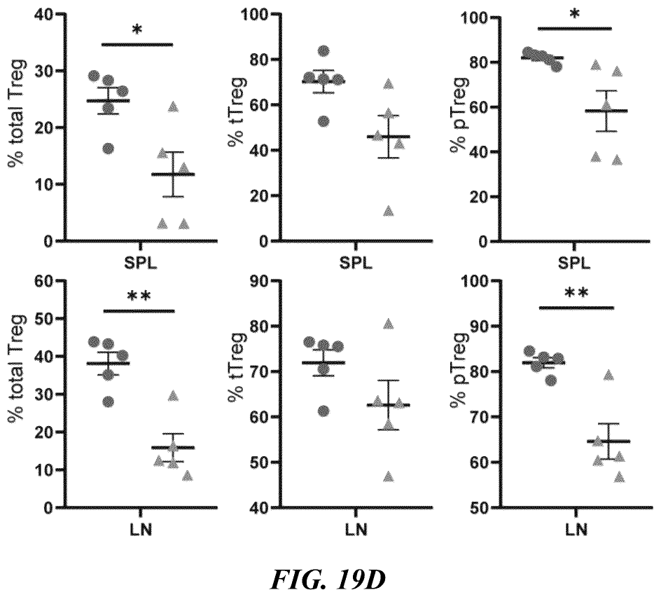

Additionally, WT and PD-L1/skin graft implanted on day 0 after HCT were compared. While all (5/5) WT STX survived for more than 100 days after HCT, 75% (6/8) of PD-L1/STX were rejected between day 20-75 after HCT (FIG. 19A). Rejection of PD-L1^{-/-}STX did not significantly impact MC levels (FIG. 19B). Rejection was associated with increased host-type CD4⁺ and CD8⁺ Tem cells in the SPL and LN (FIGS. 19C and 19D). Rejection was further associated with reduced percentage of total Treg cells among CD4⁺ Tem cells and reduced percentage of pTreg cells although no reduced percentage of tTreg cells in the SPL and LN (FIG. 19D), suggesting donor-type organ transplant expression of PD-L1 may be required for expansion of host-type pTreg cells and organ transplant tolerance in conditioning-induced MCs, although not required for induction of stable MHC-mismatched MC.

As stated above, the foregoing are merely intended to illustrate the various embodiments of the present technology. As such, the specific modifications discussed above are not to be construed as limitations on the scope of the present technology. It will be apparent to one skilled in the art that various equivalents, changes, and modifications may be made without departing from the scope of the present technology, and it is understood that such equivalent embodiments are to be included herein. All references cited herein are incorporated by reference as if fully set forth herein.

### Additional Embodiments

Various embodiments of the present technology are set forth below in paragraphs to [0209]:

1. A method of promoting or inducing organ transplant tolerance in a recipient, the method comprising
(a) administering a conditioning regimen comprising low-doses of cyclophosphamide (CY), pentostatin (PT), and anti-thymocyte globulin (ATG) to the recipient;
(b) transplanting a therapeutically effective amount of PD-L1⁺ donor-derived CD4⁺ T-depleted bone marrow cells into the recipient; and
(c) transplanting an organ into the recipient.

2. A method of promoting or inducing organ transplant tolerance in a recipient, the method comprising transplanting a therapeutically effective amount of PD-L1⁺ CD4⁺ T-depleted donor bone marrow cells into the recipient conditioned with a regimen comprising low-doses of CY, PT, and ATG to the recipient.

3. The method of embodiment 1 or 2, wherein the PD-L1⁺ donor-derived CD4⁺ T-depleted bone marrow cells include donor-derived CD4⁺ T-depleted spleen cells, and donor-derived CD4⁺ T-depleted bone marrow cells.

4. The method of embodiment 1 or embodiment 3, wherein expression of PD-L1 on the PD-L1⁺ donor-derived CD4⁺ T-depleted bone marrow cells is determined prior to the transplanting of (b).

5. The method of embodiment 1 or embodiment 3, wherein the PD-L1⁺ donor-derived CD4⁺ T-depleted bone marrow cells are selected based on PD-L1⁺ expression prior to the transplanting of (b).

6. The method of embodiment 1 or embodiment 3, wherein the PD-L1⁺ donor-derived CD4⁺ T-depleted bone marrow cells are enriched for PD-L1⁺ expression prior to the transplanting of (b).

7. The method of any one of embodiments 1 and 3-6, wherein (c) occurs before, during, or after (a) and (b).

8. The method of any one of embodiments 1 and 3-7, wherein the conditioning regimen of (a) is administered to the recipient before transplantation of the PD-L1⁺ donor-derived bone marrow cells in (b).

9. The method of any one of embodiments 1-8, wherein CY, PT, and ATG are administered simultaneously.

10. The method of any one of embodiments 1 and 3-9, wherein a population of PD-1⁺ T cells is present in the recipient before, during, or after any of (a), (b), or (c) are performed.

11. A method of promoting or inducing immune tolerance in an organ transplant recipient, the method comprising
(a) administering a conditioning regimen comprising low-doses of CY, PT, and ATG to the recipient;
(b) measuring PD-L1 expression on a population of donor-derived CD4⁺ T-depleted donor marrow cells;
(c) selecting a population of PD-L1⁺ donor-derived CD4⁺ T-depleted bone marrow cells from the population in (b); and
(d) transplanting a therapeutically effective amount of PD-L1⁺ donor-derived CD4⁺ T-depleted bone marrow cells selected in (c) into the recipient.

12. The method of any one of embodiments 2, 3, 9, and 11, wherein an organ is transplanted into the recipient.

13. The method of any one of embodiments 1-12, wherein a population of donor-derived PD-L1⁺ CD8⁺ dendritic cells is present in the recipient after organ transplant tolerance has been established.

14. The method of embodiment 13, wherein the population of donor-derived PD-L1⁺ CD8⁺ dendritic cells is derived from the transplanted bone marrow cells.

15 The method of any one of embodiments 1-14, wherein a population of recipient peripheral T regulatory cells is present in the recipient after engraftment of the transplanted bone marrow cells.

16. The method of embodiment 15, wherein the population of recipient peripheral T regulatory cells expands in the recipient after organ transplant tolerance has been established.

17. The method of any one of embodiments 1-16, wherein the administration of the conditioning regimen and transplantation of the PD-L1⁺ donor-derived CD4⁺ T-depleted bone marrow cells induces stable mixed chimerism in the recipient.

18. The method of embodiment 17, wherein the stable mixed chimerism is haploidentical stable mixed chimerism.

19. The method of any one of embodiments 1-18, wherein the donor is haploidentical to the recipient.

20. The method of any one of embodiments 1-18 wherein the donor is haplo-mismatched to the recipient.

21. The method of any one of embodiments 1-18, wherein the donor is not full-HLA- or MHC-matched to the recipient.

22 The method of any one of embodiments 1-21, wherein the donor is living or deceased.

23. The method of any one of embodiments 1-22, wherein the conditioning regimen is radiation free.

24. The method of any one of embodiments 1-23, wherein the conditioning regimen is non-myeloablative

25. The method of any one of embodiments 1-24, wherein the tolerance is immune tolerance.

26. The method of embodiment 25, wherein the immune tolerance is central immune tolerance or peripheral immune tolerance.

27. The method of embodiment 26, wherein the immune tolerance is central immune tolerance and peripheral immune tolerance.

28. The method of any one of embodiments 1-27, wherein the organ is a solid organ.

29. The method of embodiment 28, wherein the solid organ is selected from the group consisting of heart, lung, liver, kidney, intestine, pancreas, and eye.

30. The method of any one of embodiments 1-29, wherein the organ is skin.

31. The method of any one of embodiments 1-30, wherein the recipient is a human and is administered a daily dose of CY from about 25 to about 750 mg/kg/day, a daily dose of PT from about 2 mg/m²/dose to about 8 mg/m²/dose, and a dose of ATG from 1.0 mg/kg to about 8.0 mg/kg.

32. The method of embodiment 31, wherein the daily dose for CY is from about 25 mg to about 750 mg, the dose for PT is from about 2 mg/m²/dose to about 8 mg/m²/dose, and the dose for ATG is from 1.0 mg/kg to about 8.0 mg/kg.

33. The method of any one of embodiments 1-32, further comprising administration of a population of conditioning cells that facilitate engraftment during hematopoietic cell transplantation (HCT).

34. The method of embodiment 33, wherein the population of conditioning cells that facilitate engraftment during HCT is selected from one or more populations of conditioning donor cells selected from donor CD4⁺ T-depleted spleen cells, donor CD8⁺ T cells, and donor Granulocyte colony-stimulating factor (G-CSF)-mobilized peripheral blood mononuclear cells.

35. The method of embodiment 33 or embodiment 34, wherein the transplantation of the population of donor bone marrow cells occurs on the same day as or after the administration of the population of conditioning cells that facilitate engraftment during HCT.

36. The method of embodiment 35, wherein the population of conditioning donor cells, the population of donor bone marrow cells, or both are MHC- or HLA-mismatched to the recipient.

37. The method of embodiment 11, wherein the selecting in (c) comprises enriching for the population of PD-L1⁺ cells from the CD4⁺ T-depleted bone marrow cells, and optionally isolating the population of PD-L1⁺ cells from the CD4⁺ T-depleted bone marrow cells.

38. A transplant composition for transplanting into a recipient comprising a therapeutically effective amount of PD-L1⁺ donor-derived CD4⁺ T-depleted bone marrow cells and a donor organ.

39 The transplant composition of embodiment 38, wherein the transplant composition comprises a first composition and a second composition, the first composition comprising or consisting of therapeutically effective amount of PD-L1⁺ donor-derived CD4⁺ T-depleted bone marrow cells and the second composition comprising or consisting of the donor organ.

40 The transplant composition of embodiment 38 or embodiment 39, wherein expression of PD-L1 on the PD-L1⁺ donor-derived CD4⁺ T-depleted donor bone marrow cells is determined prior to the transplanting of the transplant composition into the recipient.

41. The transplant composition of any one of embodiments 38-40, wherein the PD-L1⁺ donor-derived CD4⁺ T-depleted donor bone marrow cells include donor-derived CD4⁺ T-depleted spleen cells, and donor-derived CD4⁺ T-depleted bone marrow cells.

42. The transplant composition of any one of embodiments 38 or embodiment 39, wherein the PD-L1⁺ donor-derived CD4⁺ T-depleted bone marrow cells are selected based on PD-L1⁺ expression prior to the transplanting of the transplant composition into the recipient.

43. The transplant composition of embodiment 38 or 39, wherein the PD-L1⁺ donor-derived CD4⁺ T-depleted bone marrow cells are enriched for PD-L1⁺ expression prior to the transplanting of the transplant composition into the recipient.

44. The transplant composition of any one of embodiments 38-43, wherein the donor organ is a solid organ.

45. The transplant composition of embodiment 44, wherein the solid organ is selected from the group consisting of heart, lung, liver, kidney, intestine, pancreas, and eye.

46 The transplant composition of embodiment 45, wherein the solid organ is skin.

47. The transplant composition of any one of embodiments 38-46, wherein a conditioning regimen comprising low-doses of CY, PT, and ATG is administered to the recipient.

48. The transplant composition of embodiment 47, wherein the conditioning regimen is administered to the recipient before the therapeutically effective amount of PD-L1⁺ donor-derived CD4⁺ T-depleted bone marrow cells and before the donor organ.

49. The transplant composition of embodiment 48, wherein the conditioning regimen is administered to the recipient after the therapeutically effective amount of PD-L1⁺ donor-derived CD4⁺ T-depleted bone marrow cells and before the donor organ.

50. The transplant composition of embodiment 49, wherein the conditioning regimen is administered to the recipient after the therapeutically effective amount of PD-L1⁺ donor-derived CD4⁺ T-depleted bone marrow cells and after the donor organ.

51. The transplant composition of any one of embodiments 38-50, wherein a population of recipient peripheral T regulatory cells is present in the recipient after engraftment of the transplanted bone marrow cells.

52. The transplant composition of embodiment 51, wherein the population of recipient peripheral T regulatory cells expands in the recipient after organ transplant tolerance has been established.

53. The transplant composition of any one of embodiments 38-52, wherein the donor is haploidentical to the recipient, haplo-mismatched to the recipient, or is not full-HLA- or MHC-matched to the recipient.

54. Use of the transplant composition of any one of embodiments 38-53 to promote or induce immune tolerance in a recipient.

55. Use of the transplant composition of any one of embodiments 38-54 to promote or induce organ transplant tolerance in a recipient.

56. The use of the transplant composition of embodiment 55, wherein a population of donor-derived PD-L1⁺ CD8⁺ dendritic cells is present in the recipient after organ transplant tolerance has been established.

57. The use of the transplant composition of embodiment 56, wherein the population of donor-derived PD-L1⁺ CD8⁺ dendritic cells is derived from the transplanted bone marrow cells.

### REFERENCES

1. Stewart S, Winters G L, Fishbein M C, et al. Revision of the 1990 working formulation for the standardization of nomenclature in the diagnosis of heart rejection. J Heart Lung Transplant. 2005; 24 (11): 1710-1720.
2. Etra J W, Grzelak M J, Fidder S A J, et al. A Skin Rejection Grading System for Vascularized Composite Allotransplantation in a Preclinical Large Animal Model. Transplantation. 2019; 103 (7): 1385-1391.
3. Song Q, Wang X, Wu X, et al. Tolerogenic anti-IL-2 mAb prevents graft-versus-host disease while preserving strong graft-versus-leukemia activity. Blood. 2021; 137 (16): 2243-2255.
4. Martinez R J, Zhang N, Thomas S R, et al. Arthritogenic self-reactive CD4+ T cells acquire an FR4hiCD73hi anergic state in the presence of Foxp3+ regulatory T cells. J Immunol. 2012; 188 (1): 170-181.

1. A method of promoting or inducing organ transplant tolerance in a recipient, the method comprising
(a) administering a conditioning regimen comprising low-doses of cyclophosphamide (CY), pentostatin (PT), and anti-thymocyte globulin (ATG) to the recipient;
(b) transplanting a therapeutically effective amount of PD-L1⁺ donor-derived CD4⁺ T-depleted bone marrow cells into the recipient; and
(c) transplanting an organ into the recipient.

2. A method of promoting or inducing organ transplant tolerance in a recipient, the method comprising transplanting a therapeutically effective amount of PD-L1⁺ CD4⁺ T-depleted donor bone marrow cells into the recipient conditioned with a regimen comprising low-doses of CY, PT, and ATG to the recipient.

3. The method of claim 1, wherein the PD-L1⁺ donor-derived CD4⁺ T-depleted bone marrow cells include donor-derived CD4⁺ T-depleted spleen cells, and donor-derived CD4⁺ T-depleted bone marrow cells.

4. (canceled)

5. The method of claim 1, wherein the PD-L1⁺ donor-derived CD4⁺ T-depleted bone marrow cells are (i) selected based on PD-L1⁺ expression or (ii) enriched for PD-L1⁺ expression prior to the transplanting of (b).

6. (canceled)

7. The method of claim 5, wherein (c) occurs before, during, or after (a) and (b).

8. The method of claim 7, wherein the conditioning regimen of (a) is administered to the recipient before transplantation of the PD-L1⁺ donor-derived bone marrow cells in (b).

9. The method of claim 8, wherein CY, PT, and ATG are administered simultaneously.

10. The method of claim 9, wherein a population of PD-1+ T cells is present in the recipient before, during, or after any of (a), (b), or (c) are performed.

11. A method of promoting or inducing immune tolerance in an organ transplant recipient, the method comprising
(a) administering a conditioning regimen comprising low-doses of CY, PT, and ATG to the recipient;
(b) measuring PD-L1 expression on a population of donor-derived CD4⁺ T-depleted donor marrow cells;
(c) selecting a population of PD-L1⁺ donor-derived CD4⁺ T-depleted bone marrow cells from the population in (b); and
(d) transplanting a therapeutically effective amount of PD-L1⁺ donor-derived CD4⁺ T-depleted bone marrow cells selected in (c) into the recipient.

12. (canceled)

13. The method of claim 10, wherein a population of donor-derived PD-L1⁺ CD8⁺ dendritic cells is present in the recipient after organ transplant tolerance has been established.

14. (canceled)

15. The method of claim 13, wherein a population of recipient peripheral T regulatory cells is present in the recipient after engraftment of the transplanted bone marrow cells, and the population of recipient peripheral T regulatory cells expands in the recipient after organ transplant tolerance has been established.

16. (canceled)

17. The method of claim 15, wherein the administration of the conditioning regimen and transplantation of the PD-L1⁺ donor-derived CD4⁺ T-depleted bone marrow cells induces stable mixed chimerism in the recipient.

18. The method of claim 17, wherein the stable mixed chimerism is haploidentical stable mixed chimerism.

19. The method of claim 18, wherein the donor is (i) haploidentical, (ii) haplo-mismatched, or (iii) not full-HLA- or MHC-matched to the recipient.

20.-22. (canceled)

23. The method of claim 19, wherein the conditioning regimen is radiation free or non-myeloablative.

24.-27. (canceled)

28. The method of claim 23, wherein the organ is a solid organ selected from the group consisting of heart, lung, liver, kidney, intestine, pancreas, eye, and skin.

29. (canceled)

30. (canceled)

31. The method of claim 28, wherein the recipient is a human and is administered a daily dose of CY from about 25 to about 750 mg/kg/day, a daily dose of PT from about 2 mg/m²/dose to about 8 mg/m²/dose, and a dose of ATG from 1.0 mg/kg to about 8.0 mg/kg.

32. (canceled)

33. The method of claim 31, further comprising administration of a population of conditioning cells that facilitate engraftment during hematopoietic cell transplantation (HCT), and the population of conditioning cells that facilitate engraftment during HCT is selected from one or more populations of conditioning donor cells selected from donor CD4⁺ T-depleted spleen cells, donor CD8+ T cells, and donor Granulocyte colony-stimulating factor (G-CSF)-mobilized peripheral blood mononuclear cells.

34. (canceled)

35. The method of claim 33, wherein the transplantation of the population of donor bone marrow cells occurs on the same day as or after the administration of the population of conditioning cells that facilitate engraftment during HCT.

36. The method of claim 35, wherein the population of conditioning donor cells, the population of donor bone marrow cells, or both are MHC- or HLA-mismatched to the recipient.

37.-57. (canceled)
